(19) United States
(12) Patent Application Publication
(10) Pub. No.: US 2009/0239888 A1
(43) Pub. Date: Sep. 24, 2009

(54) Methods for Inhibiting Amyloid Precursor Protein and Beta-Amyloid Production and Accumulation

(75) Inventors: James Malter, Madison, WI (US); Cara Westmark, Fitchburg, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: WISCONSIN ALUMNI RESEARCH FOUNDATION, Madison, WI (US)

(21) Appl. No.: 12/370,397

(22) Filed: Feb. 12, 2009

Related U.S. Application Data
(60) Provisional application No. 61/028,158, filed on Feb. 12, 2008.

(51) Int. Cl. A61K 31/4168; A61K 31/44; G01N 33/53; A61P 25/00

(52) U.S. Cl. 514/277; 514/389; 435/7.1

(57) ABSTRACT

Compositions and uses of mGluR₅ antagonists for the treatment and inhibition of amyloid precursor protein (APP), Aβ protein, and APP proteolytic products in Alzheimer's disease, Fragile X Syndrome, autism, and Down's Syndrome are provided. The invention provides methods for diagnosing Fragile X Syndrome via the assessment of Aβ₁₋₄₂ levels in blood plasma.

### CROSS REFERENCE

This application is related to and claims the benefit of priority to U.S. Provisional Application Ser. No. 61/028,158, filed Feb. 12, 2008, the disclosure of which is incorporated herein in its entirety.

### FIELD OF THE INVENTION

This invention is generally directed to methods and compositions for inhibiting production and accumulation of amyloid precursor protein (APP), beta-amyloid (Aβ), and APP proteolytic products. More specifically, methods and compounds are provided for antagonizing certain mGluR receptors as a means for reducing APP translation and Aβ accumulation. The invention also provides methods for diagnosing fragile X syndrome (FXS) in humans by measuring blood plasma levels of Aβ₁₋₄₂.

### BACKGROUND OF THE INVENTION

Alzheimer's disease (AD) is a neurodegenerative disorder characterized by senile plaques and neurofibrillary tangles. The plaques are predominantly composed of Aβ, a 39-42 amino acid peptide cleaved from APP. Sufficient levels of APP are necessary for synapse formation in the developing brain (Akaaboune et al., 2000, Mol Cell Neurosci 15: 355-367), while excess Aβ causes impaired synaptic function (Kamentz et al., 2003, Neuron 37: 925-937). Disordered synaptic transmission is also a hallmark of other neuronal disorders, such as autism, epilepsy, Down's syndrome, and fragile X mental retardation syndrome (FXS).

FXS is the most prevalent form of inherited mental retardation, affecting one in 4,000 men and one in 8,000 women. This X chromosome-linked disorder is characterized by moderate to severe mental retardation (overall IQ <70), autistic-like behavior, seizures, facial abnormalities (large, prominent ears and long, narrow face) and macroorchidism (Hagerman et al., 2002, John Hopkins University Press: 3-109). At the neuroanatomic level, FXS is distinguished by an overabundance of long, thin, tortuous dendritic spines with prominent heads and irregular dilations (Rudelli et al., 1985, Acta Neuropathol 67: 289-295; Wisniewski et al., 1991, Am J Med Genet. 38: 476-480). The increased length, density, and immature morphology of dendritic spines in FXS suggest an impairment of synaptic pruning and maturation. FXS has been found to result from loss of expression of fragile X mental retardation protein (FMRP) (Oberle et al., 1991 Science 252:1097-1102).

FMRP has been implicated in translational repression (Brown et al., 2001, Cell 107: 477-487; Miyashiro et al., 2003, Neuron 37: 417-431; Zalfa et al., 2003, Cell 112: 317-338; Laggerbauer et al., 2001, Hum Mol Genet. 10: 329-338; Li et al., 2001, Nuclei Acids Res 29: 2276-2283; Mazroui et al., 2002, Hum Mol Genet. 11: 3007-3017), and has been found, in the brain, to co-sediment with both translating polyribosomes (Stefani et al., 2004, J Neurosci 24: 7272-7276) and mRNPs (Zalfa et al., 2003, Cell 112: 317-338). FMRP contains an amino acid sequence motif (an RGG box; Bagni et al., 2005, Nat Rev Neurosci 6: 376-387) that binds to intramolecular G quartet sequences in target mRNAs (Darnell et al., 2001, Cell, 107 489-499), while another motif (the KH2 domain; Id.) has been proposed to bind to so-called kissing complex RNAs based on in vitro selection assays (Darnell et al., 2005, Genes Dev 19: 903-918). In addition, FMRP binds to uridine-rich mRNAs (Chen et al., 2003, Neuroscience 120: 1005-1017; Dolzhanskaya et al., 2003 Biochem Biophys Res Commun 305: 434-441). In aggregate, more than 500 mRNA ligands for FMRP have been identified, many with the potential to influence synaptic formation and plasticity (Brown et al., 2001, Cell 107: 477-487; Damell et al., 2001 Cell 107: 489-499).

FMRP regulates translation of mRNAs for certain synaptic proteins (Greenough et al., 2001, Proc Natl Acad Sci USA 98: 7101-7106; Todd et al., 2003, Proc Natl Acad Sci USA 100: 14374-14378). Translation of these synaptic proteins is also known to be regulated by group 1 metabotropic glutamate receptor (mGluR). FMRP mRNA and the mRNA of another synaptic protein, postsynaptic density-95 (PSP-95) contain putative G-quartets in their 3′-UTR and coding sequence, respectively (Todd et al., 2003 Proc Natl Acad Sci USA 100: 14374-14378; Schaeffer et al., 2001 EMBO J. 20: 4803-4813). Database searches revealed that APP mRNA possesses a G-quartet-like motif in the coding region (position 825-846 of the mouse sequence) embedded within a guanine-rich domain (694-846) containing several DWGG repeats. APP mRNAs (70% of APP695 and 50% of APP751/770) are associated with polyribosomes in rat brain (Denman et al., 1991 Arch Biochem Biophys 288: 29-38), suggesting that translational regulation could play an important role in APP production. Indeed, APP contains a 5′-UTR iron response element previously implicated in translation control (Rogers et al., 2002 J Biol Chem 277: 45518-45528). This experimental evidence is consistent with FMRP regulation of APP mRNA translation.

In humans, it would be expected that increased Aβ levels would predict an increased incidence of AD pathology in aged FXS individuals. But there have been no reports of an increased incidence of AD in FXS. Assessing age-related dementia in the mentally retarded is difficult, and very little is known about changes in phenotypes as FXS patients age. Definitive diagnosis has been relatively recent so elderly individuals with FXS have not been studied in significant numbers. The correlations between autism and increased levels of secreted APPβ have been shown. (Sokol et al., 2006, J Child Neurol, 444-449, Bailey et al., 2008, Int J Clin Exp Med, 338-344). And it has been reported that many FXS individuals are also autistic (Clifford et al., 2007, J Autism Dev Disord, 738-747, Sokol et al., 2006, J Child Neurol, 444-449). Thus, the increased production or altered processing of APP may contribute to the developmental disabilities observed in FXS.

Thus, there is a need in the art to develop methods and reagents for decreasing Aβ and APP production by modulating FMRP-dependent translation of APP translation. There is also a need for a reliable diagnostic marker for fragile X syndrome.

### BRIEF SUMMARY OF THE INVENTION

The invention provides pharmaceutical formulations and methods for ameliorating neurological disorders associated with synaptic dysfunction, cognitive impairment, behavioral deficits or combinations thereof, such as Alzheimer's disease (AD), Fragile X Syndrome (FXS), Down's syndrome (DS), autism and epilepsy. The pharmaceutical formulations and compositions of the invention comprise antagonists and inhibitors of a particular subtype of glutamate receptor, mGluR₅, wherein reduced activity or signaling from this glutamate receptor subtype results in decreased translation of APP. Because Aβ is produced by proteolytic cleavage of APP, reduced APP translation results in decreased proteolytic cleavage of APP and hence reduced production of Aβ. Reduction of APP, Aβ and APP proteolytic products thus provides an effective means for slowing the progression of neurological disorders caused by or associated with dysregulation of these proteins and proteolytic products.

The capacity of mGluR₅ inhibitors to decrease Aβ production is illustrated by results, set forth herein, of mutant and knock-out (KO) mouse strains. APP levels increased significantly in wild type (WT) mice after stimulation with the mGluR agonist DHPG, but not in synaptoneurosomes (SNs) or cultured neurons from FMRP knockout (KO) animals, which exhibit constitutive elevation of APP due to genetic inactivation of FMRP. APP mRNA was found to co-immunoprecipitate with FMRP in WT and resting SNs, but this interaction was lost after DHPG treatment. FMRP monomer was found to be capable of binding to the 5′ end of the G-rich sequence in the coding region of APP mRNA. These data are consistent with FMRP repressing translation of APP through an mGluR-dependent pathway. Consistent with constitutively elevated APP levels, the proteolytic products Aβ₁₋₄₀ and Aβ₁₋₄₂ are elevated in the brains of Fmr-1KO mice compared to WT.

The invention thus provides methods for modulating expression, translation, and accumulation of APP, Aβ, and APP proteolytic products. More specifically, the invention provides methods for reducing accumulation of APP or APP proteolytic products in neurons as well as methods for inhibiting FMRP dissociation from APP mRNA. The invention provides methods for antagonizing mGluR₅ receptor as a means to reduce APP expression and subsequent production of Aβ.

The invention also provides methods for treating or ameliorating neurodegenerative disorders including but not limited to Alzheimer's disease (AD), Fragile X Syndrome (FXS), autism, Down's syndrome and related neurological disorders, using mGluR₅ receptor-specific (inhibitory) compounds to reduce APP expression, translation, and accumulation. The invention includes methods for reducing cognitive decline, seizure frequency, or abnormal dendritic spine formation associated with the above-mentioned disorders.

The invention further provides methods for administering pharmaceutical compounds for treating Alzheimer's disease, Fragile X Syndrome (FXS), autism, Down's syndrome, and other forms of mental retardation. More specifically, in the inventive methods, mGluR₅ antagonists are used in pharmaceutical compositions and formulations for treating Alzheimer's disease, autism, Fragile X Syndrome (FXS), Down's syndrome, and other forms of mental retardation. The pharmaceutical formulations produced for treating Alzheimer's disease, autism, and other forms of mental retardation are also provided.

Thus, the invention provides methods for using mGluR₅ antagonists to treat Alzheimer's disease, Fragile X Syndrome (FXS), autism, Down's syndrome, and other forms of mental retardation. The invention further provides methods for administering a therapeutically effective amount of a pharmaceutical composition comprising an mGluR₅ antagonist to a human suffering from Alzheimer's disease, Fragile X Syndrome (FXS), autism, Down's syndrome, and other forms of mental retardation. In preferred embodiments of the invention, the mGluR₅ antagonist is fenobam.

In one aspect of the invention, the method comprises administering to a patient in need of such treatment an effective amount of an mGluR₅ antagonist or combinations thereof. In certain embodiments, mGluR₅ antagonists are administered at a minimal dosage effective for treatment or a maximal dosage under toxicity. For example, mGluR₅ may be administered in a dose ranging from about 8 to about 9 mg/kg body weight/day.

The invention thus provides advantageous alternatives to current methods and pharmaceutical compositions for treating devastating neurological disorders associated with synaptic dysfunction resulting in cognitive impairment and behavioral deficits, including (but not limited to) Alzheimer's disease (AD), Fragile X Syndrome (FXS), Down's syndrome, and autism. As set forth herein, translation of APP, which is cleaved to generate neurotoxic Aβ, can be repressed by antagonizing a particular subtype of glutamate receptor (mGluR₅) using antagonist. Stimulation of mGluR₅ rapidly increases translation of APP in neurons wherein excess APP is proteolytically cleaved to generate significantly elevated Aβ.

This invention further provides reagents and methods for identifying patients with fragile X mental retardation syndrome (FXS). The reagents and methods of the invention are directed to detecting reduced or altered levels of Aβ₁₋₄₂ in blood plasma/serum. Specific embodiments of the methods of the invention are adapted for detecting altered levels of Aβ₁₋₄₀, Aβ₁₋₄₂ or APP in blood and tissue samples.

These methods can also be used to detect altered or reduced Aβ₁₋₄₂ in patient blood plasma/serum. Use of the inventive methods detects FXS or other neurodegenerative disorders characterized by abnormal Aβ plaques or APP proteolytic products.

Specific preferred embodiments of the present invention will be better understood from the following more detailed description of certain preferred embodiments and the claims.

### BRIEF DESCRIPTION OF THE DRAWINGS

These and other objects and features of this invention will be better understood from the following detailed description taken in conjunction with the drawings wherein:

FIG. 1 is a graph illustrating the fold increase in APP mRNA levels following mGluR₅ activation. APP mRNA was co-immunoprecipitated with FMRP from WT and KO synaptoneurosomes (SN) in the presence or absence of the mGluR₅ receptor agonist DHPG. After 60 min treatment with DHPG, APP expression was analyzed by quantitative reverse transcription/polymerase chain reaction (RTqPCR). The results of these assays were plotted as the fold increase in APP mRNA compared to WT SN in the presence of DHPG. The data in the Figure is the average of three experiments. In the inset, FMRP was immunoprecipitated from WT SN incubated in the presence or absence of DHPG for 60 min and analyzed by western blot analysis. The data are representative of two experiments.

FIG. 2 is a graph illustrating the percentage increase of APP translation in SN upon mGluR₅ activation. Immunoprecipitated, ³⁵S-labeled APP (120 kDa band) from SN taken 15 and 60 min after mGluR₅ activation were analyzed by SDS-PAGE and plotted as a percentage of APP synthesis, n=3 repetitions. (*) Denotes significant differences (p=0.008) in samples in the presence versus in the absence of DHPG samples (at 15 min) and with p=0.016 between control at 15 min and DHPG-treated SNs at 60 min. For the control samples at 15 and 60 min, p=0.056, and for the samples at 60 min±DHPG, p=0.05.

FIG. 3 is a result from western blot analysis illustrating differential regulation of APP levels in WT and KO SN. Western blots of WT (top panel) and KO (bottom panel) SN treated in the presence or absence of DHPG (at 5, 10 and 20 min of incubation) were probed with anti-APP and anti-β-actin (control) antibodies. Data representative of three experiments were quantitated with ImageQuant software and demonstrated a 1.6- to 1.8-fold increase in APP between untreated and DHPG-stimulated WT SN at all times tested.

FIG. 4 is a western blot and graphical representation of the blot, showing that mGluR₅ specific inhibitor 2-methyl-6-(phenylethynyl)pyridine (MPEP) and protein translation inhibitor anisomycin each blocked mGluR activation of APP translation in SN. Immunoprecipitated, ³⁵S-labeled APP (120 kDa band) from WT SN not treated (Lane C) or treated with 100 μM DHPG (Lane D), 40 μM anisomycin+100 μM DHPG (Lanes A+D), anisomycin alone (Lane A), 10 μM MPEP+100 μM DHPG (Lanes M+D), or MPEP alone (Lane M) for 15 min were analyzed by SDS-PAGE and plotted as a percentage of APP synthesis. (n=3 repetitions (Lane D), n=4 (Lanes A+D and A) and n=5 (Lanes M+D and M).

FIGS. 5A and 5B depict DHPG-enhanced APP translation in WT but not Fmr-1 KO neurons. FIG. 5A are immunofluorescent confocal images of WT (top) and KO (bottom) neuronal cells incubated in the presence or absence of DHPG (at 0, 10 and 20 min incubation), hybridized with mouse anti-22C11 APP primary and anti-mouse rhodamine-conjugated secondary antibodies. The dashed rectangles encompass segments of dendrites, which are enlarged and displayed below the photos. FIG. 5B shows dendritic APP levels that were quantitated with ImageJ software and plotted as a percentage of untreated WT samples. (*) Denotes significant differences with p<0.001 between the pairs.

FIG. 6 is a graph showing the impact of MPEP on APP expression in primary mouse neuronal cells. Primary neuronal cells prepared from E17/18 embryos were cultured for 15 days prior to treatment with 10 mM MPEP for 15 min, 4 hr or 1 day. Fixed cells were stained with anti-22C11 and goat anti-mouse rhodamine-conjugated secondary antibody prior to visualization by confocal microscopy. Staining of dendritic puncta (minimum of 475 particles per treatment) was quantitated with Image J software using the Analyze Particles function. All MPEP-treated samples were highly statistically different from untreated controls to t-test analyses (p=0) and expressed as SEM.

FIGS. 7A and 7B are graphs depicting increased Aβ₁₋₄₀ and Aβ₁₋₄₂ levels in Fmr-1 KO mice. FIG. 7A shows the results obtained using soluble brain lysates from one-year old WT and Fmr-1 KO mice (FVB strain) analyzed by ELISA and plotted as a percentage of soluble Aβ compared to WT controls. Significance of these data was assessed using Student's t-tests: p=0.06 (Aβ₁₋₄₀) and p=0.001 (Aβ₁₋₄₂). FIG. 7B shows the results of guanidinium hydrochloride (GnHCl)-solublized brain lysates from one-year old WT and Fmr-1 KO mice (C57BL/6 strain) analyzed by ELISA and plotted as a percentage of GnHCl soluble Aβ compared to WT controls. Significance of these data was assessed using Student's T-tests: p<0.001 (Aβ₁₋₄₀) and p=0.39 (Aβ₁₋₄₂).

FIG. 8 are graphs showing the levels of APP/APPα in FXS human blood plasma, peripheral blood mononuclear cells (PBMC) cell lysates and culture media data. Open bars represent control samples and crossed bars represent FXS. Data are for APPα in control (n=7) and FXS (n=10) plasma and PBMC culture media (n=7 controls and 4 FXS) and APP in PBMC (n=7 controls and 5 FXS). For the plasma and PBMC culture media data, APPα is expressed as ng/mL and for the PBMC lysate measurements, APP is expressed as ng/mL/g lysate.

FIG. 9 are graphs showing the levels of Aβ₁₋₄₀ and Aβ₁₋₄₂ in FXS human blood plasma. Open bars represent control samples and crossed bars represent FXS. Aβ is expressed as pg/mL. Aβ₁₋₄₀ and Aβ₁₋₄₂ in control (n=7) and FXS (n=10) plasma.

FIG. 10 are graphs showing the levels of APP/APPα, Aβ₁₋₄₀ and Aβ₁₋₄₂ in FXS human brain. Open bars represent control samples and crossed bars represent FXS. Soluble Aβ₁₋₄₀ in hippocampus and neocortex of control and FXS brain autopsy tissue was assessed by ELISA after preparation of detergent-soluble lysates as described below in the Examples. For Aβ₁₋₄₂, p<0.004. Data shown are: (A) APP/APPα in control (n=3) and FXS (n=4) neocortex and hippocampus. (B) Soluble Aβ₁₋₄₀ in control (n=3) and FXS (n=4) neocortex and hippocampus and (C) Total Aβ₁₋₄₀ and Aβ₁₋₄₂ in control (n=3) and FXS (n=4) neocortex and hippocampus.

FIG. 11 is a chemical structure depiction for fenobam, a 3-chlorophenyl urea of heterocyclic creatinine, having the formal chemical name, N-(3-chlorophenyl)-N-(4,5-dihydro-1-methyl-4-oxo-1H-imidazole-2-yl)urea]sulfate.

FIG. 12 is a graph illustrating the effects of fenobam treatment in Tg2576 mice. Tg2576 mice were fed placebo or fenobam-supplemented lab diet for one month prior to harvesting brains for ELISA assays. For chronic dosing, the diet was supplemented with 0.2 g fenobam per kg lab diet and colored red for visual differentiation with the placebo food. Whole brain homogenates from control and fenobam-treated mice were analyzed by ELISA for Aβ₁₋₄₀ (left) and Aβ₁₋₄₂ (right). Data were plotted as ng/mL Aβ per μg soluble brain lysate. These results showed that chronic fenobam dosing reduced mouse Aβ₁₋₄₀ levels in Tg2576. The left bar in each group represents Aβ levels in mice fed with placebo food; and the right bar in each group represents Aβ levels in mice fed with fenobam-supplemented food.

FIG. 13 are graphs showing the inhibitory effects of fenobam treatment on Aβ₁₋₄₂ levels in cerebellum of Fmr-1 KO mice. Fmr-1 KO mice were fed placebo or fenobam-supplemented lab diet for two months prior to harvesting brains for ELISA assays. Homogenates of hippocampus (H), cortex (Cx) and cerebellum (Cb) were analyzed by ELISA for Aβ₁₋₄₀ and Aβ₁₋₄₂. Data were plotted as a percentage of Aβ compared to levels in control mouse hippocampus. C: control, F: Fenobam.

FIG. 14 are graphs showing the inhibitory effects of fenobam treatment on Aβ₁₋₄₂ levels in cortex of Fmr-1 KO mice. Fmr-1 KO mice were fed placebo or fenobam-supplemented lab diet for one month prior to harvesting brains for ELISA assays. Homogenates of hippocampus (H), cortex (Cx) and cerebellum (Cb) were analyzed by ELISA for Aβ₁₋₄₀ and Aβ₁₋₄₂. Data were plotted as a percentage of Aβ compared to levels in control mouse hippocampus.

FIG. 15 are graphs showing audiogenic seizure (AGS) rates and death rates for three mouse models that overproduce APP. AGS in fmr-1^{-/-} and APP_{Swe} Mice, and FRAXAD. Mice (age: P21) were exposed to 118 dB siren for 5 min and monitored for wild running (WR), AGS and respiratory arrest. Percent AGS (A) were 5% (WT females, n=22), 6% (WT males, n=17)), 38% (fmr-1^{-/-} females, n=8), 50% (fmr-1^{-/-} males, n=8), 36% (Tg2576 females, n=11), 40% (Tg2576 males, n=5), 64% (FRAXAD females, n=11), 92% (FRAXAD males, n=13), and percent death (B) were 5% (WT females, n=22), 6% (WT males, n=17), 38% (fmr-1^{-/-} females, n=8), 38% (fmr-1^{-/-} males, n=8), 36% (Tg2576 females, n=11), 40% (Tg2576 males, n=5), 55% (FRAXAD females, n=11), 62% (FRAXAD males, n=13). All transgenic and knock-out strains were statistically different from WT mice as assessed by Chi Square (χ²) analyses (p<x).

FIG. 16 is a graph showing the relative levels of APP Expression as a percentage of WT as measured by western blot analysis. Fmr-1 KO mice were crossed with APP KO mice to create dKO mice and Fmr-1 KO mice that were heterozygous for APP. The brains of 4 week old mice were immunoblotted for APP and the APP heterozygotes exhibited decreased APP levels.

FIG. 17 is a graph of Aβ₁₋₄₀ cortical levels in adult wild-type mice that received IP injections of 50 μg nonimmune IgG or anti-APP/Aβ antibody three days prior to sacrifice. Cortical Aβ₁₋₄₀ levels were measured by ELISA and found significantly reduced.

FIG. 18 is a graph of the number of marbles buried by male mice depending on genetic background.

### DETAILED DESCRIPTION OF PREFERRED EMBODIMENTS

The invention is more particularly described below and particularly in the Examples set forth herein that are intended as illustrative only since numerous modifications and variations therein will be apparent to those skilled in the art.

As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practitioner regarding the description of the invention. All documents cited herein are incorporated by reference in their entirety for all purposes.

The discovery that administration of mGluR₅ antagonists can reduce production of APP and Aβ suggests a mechanistic basis for treating neurological diseases. However, the practice of the methods of this invention is not limited to any particular explanatory mechanism, but instead depends on the effects of administering mGluR₅ antagonists for treating neurological disorders associated with synaptic dysfunction, cognitive impairment and behavioral deficits such as Alzheimer's disease (AD), Down's syndrome and autism. In additional embodiments, administration of mGluR₅ antagonists reduces onset or frequency of seizures.

Thus, in one aspect, the invention provides a method of reducing amyloid precursor protein (APP) production in a cell in a subject that has, or has a predisposition for developing, Alzheimer's disease (AD), autism, epilepsy, Down's syndrome, or fragile X mental retardation syndrome (FXS), wherein the method comprises contacting the cell in the subject with a metabotropic glutamate receptor (mGluR₅) antagonist. In certain preferred embodiments, the subject is a human. In certain other preferred embodiments, the cell is a neuronal cell.

As used herein the term “predisposed” referred to a propensity of developing a certain disease or condition, mainly as a result of genetic effects.

In another aspect, the invention relates to using mGluR₅ antagonists for treating AD, Fragile X Syndrome (FXS), Down's syndrome, autism, epilepsy, and related disorders. Modulation of mGluR₅ with an antagonist inhibits APP production and neurological phenotypes associated with AD. In a further embodiment, treatment with mGluR₅ antagonist inhibits production and accumulation of APP, Aβ and APP proteolytic products.

In preferred embodiments, the mGluR₅ antagonist is fenobam or MPEP. MPEP and fenobam are potent and highly selective noncompetitive antagonists of mGluR₅ that share an allosteric modulatory site (Walberg et al, 2006, Bioorg Med Chem Lett 16: 1142-1145; Porter et al., J Pharmacol Exp Ther 315: 711-721). MPEP reduces audiogenic seizures and anxiety phenotype in Fmr-1 KO mice (Yan et al., 2005, Neuropharmacology 49: 1053-1066), and fenobam reduces anxiety in mice and rats (and humans) (Porter et al., 2005, J Pharmacol Exp Ther 315: 711-721). Fenobam (FIG. 7) is an anxiolytic agent not generally associated with treatment of any other disease or disorder that displayed no harmful side effects when administered in therapeutically-effective dosages to humans (Pecknold et al., 1980, Curr Ther Res 274: 119-123; Pecknold et al., 1982, J Clin Psychopharmacol 2: 129-133; Lapierre et al., 1982, Curr Ther Res 31: 95-101), although in one study it displayed psycho-stimulating properties (Friedmann et al, 1980, Curr Ther Res 274: 144-151). In particular, fenobam displayed no activity toward other rat mGluR₅ (mGluR_{1a, 2, 4a, 7a, 8a}) at doses up to 10 μm (Porter et al., 2005, J Pharmacol Exp Ther 315: 711-721). It will be understood in the art that these two examples of mGluR₅ antagonist are non-limiting, and that any compound having specific mGluR₅ antagonist activity fall within the scope of the methods and pharmaceutical compositions of the invention. Additional examples of mGluR₅ antagonists suitable for use in the instant invention include 3-[(2-methyl-1,3-thiazol-4-yl)ethynyl]pyridine (MTEP), 3-fluoro-5-(5-pyridin-2-yl-2H-tetrazol-2-yl)benzonitrile and 5-[(2-methyl-1,3-thiazol-4-yl)ethynyl]-2,3′-bipyridine. Other suitable mGluR₅ antagonists include without limitation 6-methyl-2-(phenylazo)-3-pyridinol (SIB-1757); (E)-2-methyl-6-(2-phenylethenyl)pyridine (SIB-1893); 3-((2-methylthiazol-4-yl)ethynyl)benzonitrile; 2-methyl-4-((5-vinylpyridin-3-yl)ethynyl)thiazole; 4-((6-(4-fluorophenyl)pyridin-3-yl)ethynyl)-2-methylthiazole; 3-((3-bromophenyl)ethynyl)-5-methyl-1,2,4-triazine; 3-((3-chlorophenyl)ethynyl)-5-methyl-1,2,4-triazine; 5-methyl-3-(m-tolylethynyl)-1,2,4-triazine; 2-(4-(3-chlorophenyl)but-1-ynyl)-6-methylpyridine; 2-(2-(3-fluoro-5-(pyridin-3-yloxy)phenyl)-2H-tetrazol-5-yl)pyridine; 6-(3-methoxy-4-(pyridin-2-yl)phenyl)imidazo[2,1-b]thiazole; 3-amino-6-methyl-N-(6-methylpyridin-2-yl)pyrazine-2-carboxamide; 3-amino-6-chloro-N-(6-methylpyridin-2-yl)pyrazine-2-carboxamide; 3-hydroxy-6-methyl-N-(6-methylpyridin-2-yl)picolinamide; 3-(2-methylquinolin-7-yl)benzonitrile; 3-fluoro-5-(2-methylquinolin-7-yl)benzonitrile; 3-chloro-5-(2-methylquinolin-7-yl)benzonitrile; 3-methyl-5-(2-methylquinolin-7-yl)benzonitrile; 5-(2-methylquinolin-7-yl)isophthalonitrile; 3-(methoxymethyl)-5-(2-methylquinolin-7-yl)benzonitrile; 3-methoxy-5-(2-methylquinolin-7-yl)benzonitrile; 2-(3-chlorobenzyloxy)-6-methylisonicotinonitrile; 2-chloro-6-(3-chlorobenzyloxy)isonicotinonitrile; 2-chloro-6-((2-chloropyridin-4-yl)methoxy)isonicotinonitrile; 2-chloro-6-((2-methylpyridin-4-yl)methoxy)isonicotinonitrile; and 1-(5-chlorothiophen-3-yl)-3-(1-methyl-4-oxo-4,5-dihydro-1H-imidazol-2-yl)urea. The above compounds are discussed in Carroll, 2008, “Antagonists at Metabotropic Glutamate Receptor Subtype 5,” N.Y. Acad. Sci. 1141: 221-232 (doi: 10.1196/annals. 1441.015), incorporated by reference herein, specifically with regard to structure-activity relationships and mGluR₅ antagonist specificity.

As used herein, a compound that is a “pharmacokinetic antagonist” effectively reduces the concentration of the active drug at its site of action, e.g., by increasing the rate of metabolic degradation of the active ligand. Antagonism by receptor-block involves two important mechanisms: reversible competitive antagonism and irreversible, or non-equilibrium, competitive antagonism. As used herein, a “competitive antagonist” is a molecule which binds directly to the receptor or ligand in a manner that sterically interferes with the interaction of the ligand with the receptor. Non-competitive antagonism describes a situation where the antagonist does not compete directly with ligand binding at the receptor, but instead blocks a point in the signal transduction pathway subsequent to receptor activation by the ligand. Physiological antagonism loosely describes the interaction of two substances whose opposing actions in the body tend to cancel each other out.

The terms “effective amount” or “therapeutically effective amount” refer to the amount of a compound including an mGluR₅ antagonist that is effective, upon single or multiple dose administration to a patient, in treating the patient suffering from the named disorder. The term “treatment” or “treating” is intended to encompass also prophylaxis, therapy and cure.

A “patient” or “subject” to be treated by the subject method can mean either a human or non-human animal.

An “agonist” is a molecule that activates a certain type of receptor. By contrast, an “antagonist” is a molecule that prevents or reduces the effects exerted by an agonist on a receptor.

In another aspect, the invention provides pharmaceutical preparations comprising mGluR₅ antagonists adapted for use in the inventive methods disclosed herein. mGluR₅ antagonists for use in the methods of the invention can be conveniently formulated for administration with a biologically acceptable, non-pyrogenic, and/or sterile medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the mGluR₅ antagonists, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

Pharmaceutical formulations of the present invention can also include veterinary compositions, e.g., pharmaceutical preparations of the mGluR₅ antagonist suitable for veterinary uses, e.g., for the treatment of mammals and domestic animals.

The pharmaceutical compositions, formulations and preparations of the invention can be given orally, parenterally, topically, or rectally. They are, of course, given by forms suitable for the desired administration route. For example, they can be administered in tablets or capsule form, by injection, inhalation, ointment, suppository, controlled release patch, administration by injection, infusion or inhalation; topical by lotion or ointment; and by rectal suppositories. Oral and topical administrations are preferred.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. The selected dosage level will depend upon a variety of factors including the activity of the particular compound employed, or the ester, salt or amide thereof, the route of administration, the time of administration, the rate of excretion of the particular compound being employed, the duration of the treatment, other drugs, compounds and/or materials used in combination with the particular reuptake inhibitors employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

If desired, the effective daily dose of the active compound may be administered as two, three, four, five, six or more sub-doses administered separately at appropriate intervals throughout the day, optionally, in unit dosage forms.

While it is possible for a compound of the invention to be administered alone, it is preferable to administer the compound as a pharmaceutical formulation (composition). Pharmaceutical compositions according to the invention may be formulated for administration in any convenient way for use in human or veterinary medicine.

Thus, another aspect of the invention provides pharmaceutically-acceptable compositions or formulations comprising a therapeutically effective amount of one or more of the mGluR₅ antagonist compounds described herein, formulated together with one or more pharmaceutically acceptable carriers (additives) and/or diluents. As described in detail herein, pharmaceutical compositions of the invention can be specially formulated for administration in solid, liquid, or sustained release form, including those adapted for the following: (1) oral administration, for example, drenches (aqueous or non-aqueous solutions or suspensions), tablets, boluses, powders, granules, and pastes for application to the tongue; (2) parenteral administration, for example, by subcutaneous, intramuscular, or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile liquid.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary, paste, or a sustained released or delayed release formulation.

In other embodiments, method for diagnosing fragile X mental retardation syndrome are provided, wherein reduced blood plasma levels of Aβ₁₋₄₂ is an indicator of FXS. In certain advantageous embodiments, the levels of reduction of blood plasma levels of Aβ₁₋₄₂ as an indicator of FXS is from 1.5- to 5-fold, preferably at least 2-fold, and more preferably 2.1-fold, as compared to control.

Typically, FXS is diagnosed in children 18 to 36 months of age. Early detection is rare because unlike other congenital diseases, FXS testing in newborns is not mandatory, and the current diagnostic method is expensive. Early detection and early invention improves outcome of the disease. A mother can be asymptomatic and carry permutation (55-200 CGG repeats) on the X chromosome, and has the potential to give birth to babies with further expanded CGG repeats that constitute full mutation (more than 200 repeats) on the X chromosome. But only babies with older siblings with FXS will likely receive early FXS testing.

Thus, the invention provides an effective and less expensive method that facilitates and encourages early detection and screening of FXS in young children as well as adult. The advantages are several folds: an early FXS diagnosis in babies allows mothers to make an early decision of future pregnancy; early detection allows timely intervention, which greatly improves outcomes of the disease; and the method of diagnosing FXS of the present invention also provides a clear end-point for evaluation of the efficacy of any FXS treatments.

As described herein fragile X mental retardation protein (FMRP) binds to and represses the translation of APP mRNA (Westmark et al., 2007, PLoS Biol 5: e52). FMRP is a multi-functional mRNA binding protein that is ubiquitously expressed throughout the body with significantly higher levels in young animals (Khandjian et al., 1995, Hum Mol Genet. 4: 783-789). FMRP is regulated in the neonatal brain where it peaks at the end of the first postnatal week and declines thereafter (Lu et al., 2004, Proc Natl Acad Sci USA 101: 15201-15206). Fmr-1 mRNA and protein are down-regulated in mouse brain as a function of age with a 50% reduction between young (6 weeks) and old (60 weeks) mice (Singh et al., 2006, Mol Biol Rep 35:677-84 (Epub 2007, Sep. 7)). APP is also developmentally regulated with expression increasing during neuronal differentiation, maximal during synaptogenesis, and subsequent decline when mature connections are completed (Hung et al., 1992, Proc Natl Acad Sci USA 89: 9439-9443; Loffler et al., 1992, J Neurochem 59: 1316-1324; Masliah et al., 1992, Brain Res 593: 323-328; Moya et al., 1994, Dev Biol 161: 597-603). APP plays a critical physiological role in synapse formation and maintenance. It has been shown to promote synapse differentiation at the neuromuscular junction in Drosophila (Torroja et al., 1999, J Neurosci 19: 7793-7803) and increases the number of presynaptic terminals in transgenic mice (Mucke et al., 1994, Brain Res 666: 151-167). siRNA targeted against APP decreases presynaptic APP/APLP2 levels and reduces synaptic activity in the rat superior colliculus (Herard et al., 2006 Neurobiol Aging 27:1740-50). APP/APLP2 double knock-out mice exhibit defective neuromuscular junction (NMJ) formation, excessive nerve terminal sprouting and defective synaptic transmission (Wang et al., 2005, J Neurosci 25: 1219-1225). Administration of anti-APP antibodies prevents memory formation in day-old chicks (Mileusnic et al., 2005, Ann N Y Acad Sci 1048: 149-165) and in rats (Doyle et al., 1990, Neurosci Lett 115: 97-102). Thus, misregulated expression/processing of APP likely plays an important role in seizure induction and/or propagation.

Peak levels of FMRP occur at P7 and then decline whereas APP levels are highest during synaptogenesis. This timeframe of high APP levels coincides with the critical period of sensory development in rodents (postnatal weeks 2-6) (Berardi et al., 2000, Curr Opin Neurobiol 10: 138-145). Audiogenic seizure (AGS) sensitivity peaks at P21 in fmr-1^{-/-} mice and declines with age, and likewise human FXS patients outgrow seizures by the end of adolescence (Wisniewski et al., 1991, Am J Med Genet 38: 476-480; Berry-Kravis, 2002, Dev Med Child Neurol 44: 724-728). At the other end of the age spectrum, seizure incidence increases with old age, particularly in AD. Studies provided herein indicate that higher APP/Aβ levels correlate with increased seizure propensity.

Inhibition of mGluR₅ as described herein is a therapeutic treatment for FXS. There is an increase in mGluR₅-mediated signaling in the hippocampus of fmr-1^{-/-} mice (enhanced LTD) (Huber et al., 2002, Proc Natl Acad Sci USA 99: 7746-7750), and the chronic reduction of mGluR₅ gene dosage in these mice produces a substantial rescue of many FXS phenotypes including excessive sensitivity to environmental change, synaptic connectivity, protein synthesis, memory extinction, body growth and excitability in the form of AGS (Dolen et al., 2007, Neuron 56: 955-962). MPEP is a potent and highly selective noncompetitive antagonist of mGluR₅ (Wallberg et al., 2006, Bioorg Med Chem Lett 16: 1142-1145; Porter et al., 2005, J Pharmacol Exp Ther 315: 711-721) and intraperitoneal (IP) injections of the drug reduce AGS and anxiety phenotypes in fmr-1^{-/-} mice (Yan et al., 2005, Neuropharmacology 49: 1053-1066). Herein, a strong AGS phenotype was attenuated by pre-treatment with MPEP in Tg2576 and FRAXAD mice.

Studies described in the Examples characterize several mouse models that overproduce APP or its metabolites. The seizure threshold in fmr-1^{-/-}, Tg2576, FRAXAD and DS mice was measured in Examples 6 & 7. PTZ- and audiogenic-induced seizures both mimic primary tonic-clonic human epilepsy (Bradford, 1995, Prog Neurobiol 47: 477-511), however, their physiological basis differs. PTZ-induced seizures are mainly generated and propagated through the hippocampus whereas AGS requires activation of brain auditory pathways with initiation in the midbrain inferior colliculus (Chakravarty et al., 1999, Exp Neurol 157: 135-141; Kwon et al., 1997, Epilepsy Res 27: 89-99; Novak et al., 1989, Brain Res 497: 223-230) as well as the hippocampus (Reid et al., 1983, Exp Neurol 82: 237-240). Central auditory function is also impaired in AD where neuritic plaque formation is found in the ventral nucleus of the medial geniculate body (MGB) and the central and dorsomedial nuclei of the inferior colliculus (IC) as well as the deep layers of the dorsal cortex (Sinha et al., 1993, Neurology 43: 779-785; Ohm et al., 1989, Neurosci Lett 96: 60-63). The ventral nucleus of the MGB, the major thalamic relay station for auditory function, receives fibers from neurons of the central nucleus of the IC, which is the largest structure in the auditory brainstem pathway and processes both ascending and descending information. Thus, the over-expression of APP or proteolytic products of APP in these brain stem regions contributes to sensory hyper-reactivity. Despite differences in brain location for the generation and propagation of these two distinct seizure paradigms (PTZ and AGS), these studies suggest that APP/sAPP and/or Aβ are a common denominator at the molecular level. Over-expression of APP and/or catabolites lowers the threshold to both PTZ- and audiogenic-induced seizures.

Other mouse models suitable for studying induced seizures include (1) APP23 mice, which over-express hAPP_{751swe} (Lalonde et al., 2005, Behav Brain Res 157: 91-98), (2) CRND8, which over-express the Swedish and Indiana familial AD mutations in APP₆₉₅ (Del Vecchio et al., 2004, Neurosci Lett 367: 164-167), (3) mice with a double mutation in the α-secretase site (Moechars et al., 1996, EMBO J. 15: 1265-1274), (4) APP^{-/-} mice (Steinbach et al., 1998, Cell Death Differ 5: 858-866), (5) BACE^{-/-}PDAPP⁻ mice (Kobayashi et al., 2008, Neurobiol Aging 29: 861-873), (6) BACE^{-/-}PDAPP⁺ mice (Kobayashi et al., 2008, Neurobiol Aging 29: 861-873), (7) Tg2576 mice (Westmark et al., 2008, Int J Clin Exp Pathol 1: 157-168), which over-express the coding region of hAPP_{Swe} resulting in a 5-fold increase in hAβ₁₋₄₀ and a 14-fold increase in hAβ₁₋₄₂ (Hsiao et al., 1996, Science 274: 99-102), (8) FRAXAD mice, which are a cross between fmr-1^{-/-} and Tg2576 mice (Westmark et al., 2008, Int J Clin Exp Pathol 1: 157-168), and (9) hAPP-J20 transgenic mice, which carry the Swedish and Indiana familial mutations (Palop et al., 2007, Neuron 55: 697-711).

Embodiments of the methods of this invention comprising the above-mentioned features are intended to fall within the scope of the invention.

### EXAMPLES

The Examples that follow are illustrative of specific embodiments of the invention, and various uses thereof. They set forth for explanatory purposes only, and are not to be taken as limiting the invention.

### Materials and Methods for Examples 1-3

Anti-FMRP antibody (mAb7G1-1) was obtained from the Developmental Studies Hybridoma Bank, University of Iowa (http://www.uiowa.edu/˜dshbwww). Anti-APP polyclonal antisera (catalog number 51-2700) was purchased from Zymed Laboratories (http://www.invitrogen.com), and anti-mouse β-actin antibody (catalog number A5441), protease inhibitor cocktail (catalog number P2714), ribonuclease T1 (catalog number R1003), and poly(D)-lysine (catalog number P6407) were purchased from Sigma Chemical Company (http://www.sigmaaldrich.com). Anti-rabbit and anti-mouse HRP-conjugated secondary antibodies, Percoll®, Redivue Pro-Mix-L [³⁵S] (catalog number AGQ0080) and enhanced chemiluminescence detection reagents were obtained from Amersham Pharmacia (http://www5.amershambiosciences.com). Mouse anti-22C11 APP monoclonal antibody (mAB348) was acquired from Chemicon (http://www.chemicon.com). Rabbit polyclonal Aβ₁₋₄₀ (catalog number 9131), Aβ₁₋₄₂ (catalog number 9134), and rodent Aβ (catalog number 9154) antisera were purchased from Signet Laboratories (http://www.signetlabs.com). (R,S)-3,5-dihydroxyphenylglycine (DHPG) (catalog number 0805) was obtained from Tocris Cookson (http://www.tocris.com). Omniscript RT was obtained from Qiagen (http://www.qiagen.com). MagnaBind Protein A beads, PAGEprep advance kit, and micro BCA protein assay reagent kit were obtained from Pierce Biotechnology (http://www.piercenetcom). DNA oligonucleotides were custom synthesized by Integrated DNA Technologies (http://www.idtdna.com), and SYBR Green PCR master mix was obtained from Applied Biosystems (http://www.appliedbiosystems.com). NeuroBasal medium, B27 supplement, goat anti-mouse rhodamine-conjugated antibody, and ProLong Gold Antifade with DAPI were from Invitrogen (http://www.invitrogen.com). TRI-Reagent was purchased from Molecular Research Center (http://www.mrcgene.com). MPEP was purchased from Tocris Cookson or synthesized by Technically (http://www.technically.com) and provided by FRAXA Research Foundation (http://www.fraxa.org).

Mouse husbandry. The WT and Fmr-1 KO mice in the FVB and C57BL/6 backgrounds were a generous gift from Aaron Grossman and Dr. Bill Greenough (University of Illinois at Urbana-Champaign). The Fmr-1 KO mice were originally developed by Frank Kooy and backcrossed >11 times to FVB mice, albeit these FVB mice have the genes for pigmentation and normal vision (1994, Cell 78: 22-23). Mice were housed two to four animals per microisolator cage on a 6 am-6 pm light cycle using an ad libitum water and feeding (Purina 5015 mouse diet; http://www.purina.com) schedule. Cages contained seeds and a neslet as the only sources of environmental enrichment.

WT, fmr-1^{-/-}, Tg2576 and FRAXAD mice were generated, bred and housed as previously described (Westmark, et al., 2008, Int J Clin Exp Pathol 1, 157-168). All of these strains were in the C57BL/6 background (backcross n=5 or greater). APP_{Swe} and fmr-1 KO genotypes were determined by PCR analysis of DNA extracted from tail biopsies. The Ts65Dn mice were generated by crossing Ts65Dn females (Jackson Laboratories catalog #001924) with WT B6EiC3SnF1/J males (Jackson Laboratories catalog #001875) resulting in WT and trisomic offspring in a mixed background. Offspring were genotyped by quantitative real time PCR with TaqMan TAMRA probes against APP and ApoB as described by Don Liu and colleagues on the Jackson Laboratories webpage (http://www.jax.org/cyto/quanpcr.html) with the trisomic mice having 1.5-fold greater levels of the APP gene than controls.

Adequate measures were taken to minimize pain or discomfort to the mice, and all husbandry, seizure and euthanasia procedures were performed in accordance with NIH guidelines and an approved University of Wisconsin Madison animal care protocol administered through the Research Animal Resources Center.

Audiogenic Seizure Testing, Data Analyses and mGluR5 Blockade: All mouse strains were tested at postnatal day 21, the peak of AGS sensitivity. The experimental apparatus consisted of a clear, Plexiglas box (13″L×8″W×7″H) with the sound source located inside the box (LOUD KEY™ jogger's alarm). Mice were weighed and then placed individually into the center of the chamber and exposed to a siren that generated noise at 118 dB for 5 min. Loud, acoustic stimulation causes wild running (WR) behavior within 20-30 sec followed by erratic leaping, clonic convulsions and tonic hind limb extension by 40-50 sec followed by respiratory arrest and death (Chen, et al., 2001, Neuroscience 103, 1043-1050). The animals were scored for the latency time of wild running (WR), AGS and respiratory arrest (death). The percentage of mice exhibiting these phenotypes was plotted versus gender and genotype and assessed for statistical significance by Chi-square (χ²) analyses (http://www.graphpad.com/quickcalcs/chisquared1.cfm). MPEP was a kind gift from FRAXA Research Foundation (Newburyport, Mass.) and was dissolved at 1 mg/mL in DPBS before I.P. injection at 30 mg/kg body weight 30 min prior to AGS testing.

SN preparation and stimulation. SNs were prepared from WT and Fmr-1 KO mouse cortical tissue (Dunkley et al., 1988, Brain Res 441: 59-71; Bagni et al., 2000, J Neuorsci 20: RC76). Briefly, mouse pups aged 14-17 d were killed by carbon dioxide asphyxiation followed by removal of the brain cortices. The cortices were washed in ice-cold gradient medium (GM buffer: 0.25 M sucrose, 5 mM Tris (pH 7.5), and 0.1 mM EDTA), transferred to a glass dounce homogenizer containing ice-cold GM buffer, and gently homogenized with five strokes of the loose pestle followed by five strokes of the tight pestle. The homogenate was spun at 1000 g for 10 min at 4° C. in round-bottom tubes to pellet cellular debris and nuclei. The supernatant (2 ml aliquots) was applied to Percoll® gradients (layers were 2 ml each comprising sequentially 23%, 15%, 10%, and 3% isomotic Percoll®) and centrifugation performed at 32,500 g for 5 min at 4° C. The third band from the top of the gradient (at about the 23%/15% interface) containing intact SNs was removed and pooled for the experiments disclosed herein. The two higher-molecular-weight bands at the 15%/10% and 10%/3% interfaces contain broken membranes. The salt concentration of the SNs was adjusted by adding one-tenth volume of 10× stimulation buffer (100 mM Tris (pH 7.5), 5 mM Na₂HPO₄, 4 mM KH₂PO₄, 40 mM NaHCO₃, 800 mM NaCl). To suppress nonspecific excitation, 1 μM tetrodotoxin was added. The protein concentration of the SNs was determined by Bradford assay and ranged from 200-500 ng/μl.

SNs were equilibrated to room temperature by rotation on a nutator mixer for a minimum of 10 min. DHPG was dissolved in 1× stimulation buffer immediately prior to use and added to the SNs (100 μM final concentration). Samples were mixed at room temperature in 1.5 ml Eppendorf tubes for the indicated times.

Radiolabeling SNs with ³⁵S-Met and immunoprecipitation of APP. WT and KO SNs (450 μl) were mixed with 25 μl Redivue Pro-Mix-L [³⁵S] for 5 min prior to stimulation with 25 μl 2 mM DHPG. Samples were flash frozen at the indicated times. To analyze new protein synthesis, SN lysates were cleared of free isotope, Percoll®, and sucrose by purification with the PAGEprep Advance kit according to the manufacturer's directions. Protein concentrations were determined by the BCA assay, and 15 μg protein was denatured and loaded per lane on 12% SDS gels for content analysis. Gels were dried and exposed to a phosphorimager screen to identify the results of these assays.

To specifically analyze APP synthesis, WT and KO SN lysates (500 μl) were immunoprecipitated with APP antibody. Briefly, SN lysates were precleared with protein A magnetic beads in 1 ml volumes containing 500 μl SNs, 500 μl 2×IP buffer (20 mM HEPES (pH 7.4), 400 mM NaCl, 60 mM EDTA (pH 8), and 2% Triton X-100), protease inhibitor cocktail, and 100 μl packed fresh protein A magnetic beads. For the immunoprecipitations, 10 μg anti-APP antibody (Zymed catalog number 51-2700) and fresh protein A magnetic beads were added and mixed overnight at 4° C. The beads were washed three times with IP buffer, and the final, washed pellets were suspended in 40 μl 2×SDS sample buffer and boiled for 5 min; the proteins were then separated on 12% SDS gels. The gels were transferred to a nitrocelluose membrane, dried, exposed to a phosphorimager screen, and scanned on a STORM 860 phosphorimager (Molecular Dynamics http://www6.amershambiosciences.com). APP bands at 120 kDa were quantitated with ImageQuant software (GE Healthcare Life Sciences, http://www4.amershambiosciences.com).

For mGluR₅ inhibitor studies, SNs (425 μl) were preincubated with 25 μl anisomycin (40 μM final concentration) or MPEP (10 μM final concentration) for 10 min prior to the addition of 25 μl ³⁵S-Met for 5 min and stimulation with DHPG (100 μM final concentration) for 15 min. Samples were processed as described in the preceding paragraph.

Western blot analysis. Aliquots of SNs were collected at 5, 10, and 20 min after DHPG treatment, quenched with an equal volume of 2×SDS sample buffer (8% SDS, 24% glycerol, 100 mM Tris (pH 6.8), 4% β-mercaptoethanol, 0.02% bromophenol blue, 2% Triton X-100, 2% deoxycholate, 2% NP-40 alternative, and 2% sarkosyl) and boiled for 5 min prior to analysis by 12% SDS-PAGE. The separated proteins were transferred to a 0.45 μm nitrocellulose membrane in Towbin buffer containing 20% methanol with a Criterion Blotter (Bio-Rad, http://www.bio-rad.com; 100 V, 4° C., 75 min). The membranes were blocked in 5% nonfat dry milk and hybridized with anti-rabbit APP antibody (dilution, 1 μg/ml) and anti-mouse β-actin antibody (dilution, 1:20,000) followed by hybridization with anti-rabbit or anti-mouse HRP-conjugated secondary antibodies (dilution, 1:2000). Proteins were visualized by enhanced chemiluminescence on a STORM 860 phosphorimager.

Neuronal cell culture, confocal microscopy, and image analysis. Pregnant female mice at embryonic day 18 (E18) were anesthetized with halothane prior to decapitation and transfer of the uterine sac to ice-cold HBSS. Cortices were removed, washed with ice-cold HBSS, lysed with 0.5 mg/ml trypsin for 25 min at 37° C., washed with HBSS, suspended in NeuroBasal medium (supplemented with 2% B27 supplement, penicillin/streptomycin, and 0.5 mM glutamine), triturated 70× with a 10-ml pipet, and passed through a 70-μm cell strainer. Cells were counted by trypan blue dye exclusion and plated at 1.25×10⁵ cells/ml on poly(D)-lysine-coated glass coverslips in 12-well tissue-culture dishes and cultured for 11 d at 37° C./5% CO₂. Half of the culture medium was removed and replaced with fresh, warm medium on day 4.

Neuronal cells were treated with 100 μM DHPG, washed with PBS containing 2% FBS, fixed in 4% PHA for 10 min at room temperature, and permeabilized with methanol (−20° C.) for 15 min. Fixed, permeabilized cells were stained with anti-22C11 monoclonal antibody against the amino-terminus of APP (Chemicon number mAB348; 1:2000 for 1 h) and visualized with goat anti-mouse rhodamine-conjugated secondary antibody (Invitrogen; 1:500 for 30 min in the dark). All washes and antibody dilutions were in PBS containing 2% FBS. Coverslips were fixed to slides with 12 μl ProLong Gold Antifade with DAPI (Invitrogen) and dried overnight.

Images were acquired with a Nikon C1 laser-scanning confocal microscope with EZ-C1 v2.20 software (Nikon, http://www.nikon.com) at 60× magnification. APP levels in the puncta of four to seven dendrites per sample were quantitated with IMAGE J software using the Analyze Particles function (minimum of 205 puncta analyzed per treatment) (Rasband, Image J, U.S. National Institutes of Health, http://rsb.info.nih.gov/ij; 1997-2006). Figures were assembled with Adobe Photoshop 8.0 (Adobe Systems, http://www.adobe.com). All DHPG-treated and Fmr-1 KO samples were found to be highly statistically different from untreated WT samples by Student's t-test analyses (p<0.001) and expressed as SEM.

APP mRNA measurements. Aliquots of SNs were collected at the indicated timepoints and flash frozen at −80° C. Samples were thawed and vortexed to prepare SN lysates. To directly reverse-transcribe RNA from SN lysates without an RNA purification step, a modified method for detecting mRNA in single neurons was used (Cormer et al., 1999 Brain Res Brain Res Protoc 4: 367-377). Briefly, 2 μl SN lysate was added per standard reverse transcriptase (RT) reaction containing RNase-free DNase I and random nonamer primer. Reactions were incubated at 37° C. for 15 min to destroy any contaminating genomic DNA, 65° C. for 5 min to inactivate the DNase I, and 20° C. for 10 min to anneal the random primer. Omniscript RT was added and reverse transcription proceeded at 37° C. for 60 min before inactivation at 93° C. for 5 min. The RT reactions were diluted 5-fold with DEPC water prior to real-time PCR analysis. For statistical analysis, APP mRNA levels from triplicate experiments were determined, normalized to 18S rRNA, and plotted as a percentage of total APP mRNA. Data for these experiments is reported with error bars depicting the standard error of the mean (SEM).

Real-time PCR controls, standard curves, and analyses. PCR primers were designed with Primer Express software from Applied Biosystems, and BLAST homology searches of the amplicons revealed that the primers were gene specific. PCR reactions were optimized for primer and template concentrations and contained 500 nM APP primers (forward: 1701-ccgtggcacccttttgg-1717 (SEQ ID NO: 1); and reverse: 1774-gggcgggcgtcaaca-1760 (SEQ ID NO: 2) or 300 nM 18S primers (forward: 98-cattaaatcagttatggttcctttgg-123 (SEQ ID NO: 3); and reverse: 181-tcggcatgtattagctctagaattacc⁻¹⁵⁵ (SEQ ID NO: 4), 10.5 μl diluted (1:5) RT reaction and 12.5 μl SYBR green PCR mix in a 25 μl reaction volume. PCR cycle conditions were as follows: 2 min at 50° C. and 10 min at 95° C. (40 cycles: 15 s at 95° C., 1 min at 60° C.), followed by a dissociation stage for 15 s at 95° C., 1 min at 60° C., and 15 s at 95° C. The average PCR efficiencies for the APP and 18S primers over a 200-fold concentration range were 100% (APP) and 101% (18S) (n=9 experiments each), with a delta slope of 0.079. As the difference in slopes between the sample PCR (APP) and the normalization control (18S) was less than 0.1, the comparative CT method was utilized to calculate the relative concentration of APP mRNA normalized to 18S rRNA. SNs are void of nuclei; however to ensure there was no genomic DNA contamination, control RT reactions on SN templates in the absence of reverse transcriptase were analyzed by real-time PCR and found void of APP PCR product. The final APP and 18S PCR products were analyzed by agarose gel electrophoresis and were single bands of the correct molecular weight detected by ethidium bromide (EtBr) staining (74 bp for APP; 84 bp for 18S).

FMRP IPs and real-time PCR analysis. SN lysates were precleared with protein A magnetic beads and immunoprecipitated with 10 μl RNasin, 10 μg 7G1-1 antiFMRP antibody (or in the absence of antibody as a control), and 100 μl packed fresh protein A magnetic beads for 3 h at 4° C. The immunoprecipitated lysate components (IPs) were washed with IP buffer (10 mM HEPES (pH 7.4), 200 mM NaCl, 30 mM EDTA (pH 8), and 0.5% Triton X-100) and suspended in 1 ml TRI-Reagent. Total RNA was isolated and precipitated in the presence of 2 μg tRNA. The final pellet was suspended in DEPC-treated water, solubilized 10 min at 60° C., and reverse transcribed with Qiagen Omniscript and random nonamer primer (60 min at 37° C., 5 min at 93° C.). cDNA produced in this reaction was diluted 5-fold and analyzed for APP by qPCR as described immediately above.

Preparation of whole-cortex lysate. The cortices from six WT mice (13 d old) were torn into pieces and homogenized in cold immunoprecipitation buffer (10 mM HEPES (pH 7.4), 200 mM NaCl, 30 mM EDTA (pH 8), and 0.5% Triton X-100) containing 2× protease inhibitor cocktail and 0.4 U/μl RNasin. The homogenate was subjected to centrifugation at 1,000 g for 10 min at 4° C. to remove nuclei and unlysed cells, and the pellet was discarded. The cleared lysate was flash frozen in aliquots at −80° C.

Ribonuclease T1 digestions and modified CLIP assay: Pellets from anti-FMRP1 antibody immunoprecipitations of whole-cortex lysate were washed once with immunoprecipitation buffer and once with DPBS before digestion with ribonuclease T1 (0.8-4.0 U) in a 100-μl reaction volume for 30 min at 37° C. with occasional mixing to disperse the magnetic protein A beads. The digested samples were washed twice with DPBS to remove RNA fragments. Protected RNA was isolated with TRI-Reagent and analyzed by RTqPCR. The primer sequences for the real time PCR are listed in Table 1. The delta Cₜ between undigested and T1-digested samples was calculated and plotted as a percentage of APP₆₉₉₋₇₉₆ mRNA.

TABLE 1

| | | | | |
|---|---|---|---|---|
| | | | | amplicon |
| | | Reverse | | length |
| Forward Primer | Sequence | Primer | Sequence | (nt) |

| | | | | |
|---|---|---|---|---|
| APP 446-463 | 5′-ccttctcgtgcccgacaa-3′ | APP 518-497 | 5′- | 73 |
| | (SEQ ID NO: 5) | | ccactgaagatgggtc | |
| | | | tcacaa-3′ (SEQ | |
| | | | ID NO: 6) | |
| APP 584-598 | 5′-gccctgcggcatcga-3′ | APP 658-641 | 5′- | 75 |
| | (SEQ ID NO: 7) | | tccacgctgtcgctttc | |
| | | | c-3′ (SEQ ID | |
| | | | NO: 8) | |
| APP 699-719 | 5′- | APP 796-774 | 5′- | 98 |
| | ggtggagcggacacagactac-3′ | | tacgcttcctcttcctca | |
| | (SEQ ID NO: 9) | | acatc-3′ SEQ ID | |
| | | | NO: 10) | |
| APP 774-797 | 5′- | APP 871-852 | 5′- | 98 |
| | gatgttgaggaagaggaagctgat- | | ctctcggtggcctcttc | |
| | 3′ (SEQ ID NO: 11) | | gta-3′ (SEQ ID | |
| | | | NO: 12) | |
| APP 774-797 | 5′- | APP 888-869 | 5′- | 115 |
| | gatgttgaggaagaggaagctgat- | | tggcagtgctggttgtt | |
| | 3′ (SEQ ID NO: 13) | | ctc-3′ (SEQ ID | |
| | | | NO: 14) | |
| APP 757-779 | 5′- | APP 871-852 | 5′- | 115 |
| | aagaggaggaagtggctgatgtt- | | ctctcggtggcctcttc | |
| | 3′ (SEQ ID NO: 15) | | gta-3′ (SEQ ID | |
| | | | NO: 16) | |
| APP 757-779 | 5′- | APP 888-869 | 5′- | 132 |
| | aagaggaggaagtggctgatgtt- | | tggcagtgctggttgtt | |
| | 3′ (SEQ ID NO: 17) | | ctc-3′ (SEQ ID | |
| | | | NO: 18) | |
| APP 774-797 | 5′- | APP 915-896 | 5′- | 142 |
| | gatgttgaggaagaggaagctgat- | | cggactcagtggtggt | |
| | 3′ (SEQ ID NO: 19) | | tgtg-3′ (SEQ ID | |
| | | | NO: 20) | |
| APP 757-779 | 5′- | APP 915-896 | 5′- | 159 |
| | aagaggaggaagtggctgatgtt- | | cggactcagtggtggt | |
| | 3′ (SEQ ID NO: 21) | | tgtg-3′ (SEQ ID | |
| | | | NO: 22) | |
| APP 2318-2347 | 5′- | APP 2416- | 5′- | 99 |
| | gcctgaacttgaattaatatacaaat | 2392 | cagtacacaaaaccca | |
| | cagt-3′ (SEQ ID NO: 23) | | tgaatcatg-3′ | |
| | | | (SEQ ID NO: | |
| | | | 24) | |

* Nucleotide reference numbering based on mouse APP gene sequences, GenBanik Accession No. X59379

For the modified CLIP (Cross-Link and ImmunoPrecipitation) assay (Ule et al., 2003 Science 302: 1212-1215), SN were cross-linked with 400 mJ/cm² ultraviolet light in an UV Stratalinker 2400 (Stratagene, http://www.stratagene.com), immunoprecipitated with anti-FMRP antibody, and digested with ribonuclease T1. The washed pellets were suspended in 40 μl SDS loading buffer containing no reducing agents, heated for 10 min at 70° C., applied to 12% SDS/PA gels, and transferred to a 0.45 μm nitrocellulose membrane in Towbin buffer. Western blot analysis of a duplicate membrane indicated that FMRP migrates at 80 kDa. A band encompassing approximately the 75-85 kDa molecular weight range was excised, transferred to TRI-Reagent, and vortexed vigorously for 15 min at 37° C. RNA was isolated and analyzed by RTqPCR.

Aβ₁₋₄₀ and Aβ₁₋₄₂ ELISAs: For soluble brain lysates, right hemispheres from four WT (aged 11, 13, 13, and 13 mo) and three KO mice (aged 11, 12, and 12 mo; FVB strain) and four WT (aged 13.5, 13.5, 12, and 12 mo) and four KO mice (aged 14, 14, 12, and 12 mo; C57BL/6 strain) were homogenized in 500 μl protein extraction buffer (10 mM Tris (pH 7.6), 2 mM EDTA, 150 mM NaCl, 1% Triton X-100, 0.25% NP-40, and 1× protease inhibitor cocktail). Insoluble material was removed by centrifugation at 12,000 rpm for 10 min, and aliquots of the soluble fraction were flash frozen. For total brain lysates, left hemispheres were homogenized in cold, 5 M GnHCl, mixed for 3-4 h at room temperature, and frozen at −80° C. Sandwich ELISAs with the Signet Aβ₁₋₄₀/9131 and Aβ₁₋₄₂/9134 capture antibodies and the rodent Aβ/9154 reporter antibody were performed as previously described (Constantini et al., 2005 Biochem J 391: 59-67). Aβ levels were quantified based upon standard curves from experiments performed on the same ELISA plate and then expressed as a percentage of Aβ compared to WT controls.

Collection and Analysis of Human Samples: FXS and age-matched control males were recruited from the FXS Clinic at Rush University Medical Center (RUMC) in Chicago, Ill. The study was approved by the RUMC Institutional Review Board and all donors or their legal guardians signed the appropriate consent forms for study participation. All FXS subjects (ages 9-32 years old) were positive by DNA analyses for a fully methylated expansion mutation in the FMR-1 gene. Controls (age 23-33) were normal volunteers working at RUMC and had no history of cognitive or mental health disorders. Blood plasma and peripheral blood mononuclear cells (PBMC) were analyzed by ELISA for APP/APPα, Aβ₁₋₄₀ and Aβ₁₋₄₂.

These assays were performed as follows. First, the anticoagulation effect of lithium heparin, EDTA, sodium heparin and buffered sodium citrate were compared and APP and Aβ levels in both plasma and PBMC were assessed. Based on these results, lithium heparin tubes were selected for subsequent blood collection and analyses of APP/APPα and Aβ. Several commercial Aβ ELISA kits are available from sources including BioSource Intl. (Camarillo, Calif.), Alpha Diagnostic Intl. (San Antonio, Tex., catalog #200-110-A42), Covance Inc. (Princeton, N.J., catalog #SIG-38942), Wako Chemicals, Inc. (Richmond, Va., catalog #298-62401).

Commercially available Aβ ELISA kits {BioSource Intl. (Camarillo, Calif.)} with detection limits <6 pg/mL for Aβ₁₋₄₀ and <10 pg/mL for Aβ₁₋₄₂ were utilized. The incubation time was increased from 3 hr at room temperature as described by the manufacturer to overnight at 4° C., which improved antigen/antibody binding. Plasma Aβ₁₋₄₂ levels can be difficult to assess as the peptide is very hydrophobic and can bind to other proteins (Kuo et al., 1999, Biochem Biophys Res Commun 257, 787-791). With denaturation of plasma samples followed by chromatographic separation and europium-immunoassay for Aβ quantification, Aβ₁₋₄₂ levels in the range of 7.1-85.7 ng/mL for controls (Kuo et al., 1999, Biochem Biophys Res Commun 257, 787-791), which is more than 20-fold higher than we observed and 1000-fold higher than other reports (Hansson et al., 2008, Neurobiol Aging, (ePub ahead of print); Mayeux et al., 2003, Neurology. 61, 1185-1190; van Oijen et al., 2006, 61, 1185-1190). Thus, both our Aβ₁₋₄₀ and Aβ₁₋₄₂ levels are within previously reported ranges.

### Example 1

### FMRP Was Associated Directly with APP mRNA

To determine whether FMRP regulated APP expression, mRNA/protein binding studies were performed. More specifically, RNase protection and modified CLIP assays were utilized to assess FMRP protein interaction with APP mRNA. The modified CLIP assay (Ule et al., 2003 Science 302: 1212-1215) demonstrated that FMRP associated directly with APP mRNA at nt 699-796, as opposed to indirect interaction for example in an mRNP complex. Pelletted material from anti-FMRP immunoprecipitations of whole-cortex lysate were washed once with immunoprecipitation buffer and once with DPBS before digestion with ribonuclease T1 (0.8-4.0 U) in a 100-μL reaction volume for 30 min at 37° C. with occasional mixing to disperse the magnetic protein A beads. The digested samples were washed twice with DPBS to remove RNA fragments. Protected RNA was isolated with TRI-Reagent and analyzed by RTqPCR. The primer sequences for real time PCR are listed in Table 1. The delta Cₜ between undigested and T1-digested samples was calculated and plotted as a percentage of APP₆₉₉₋₇₉₆ mRNA.

For the modified CLIP assay (Ule et al., 2003 Science 302: 1212-1215), cleared cortical lysate was cross-linked with 400 mJ/cm² ultraviolet light in an UV Stratalinker 2400 (Stratagene, http://www.stratagene.com), immunoprecipitated with anti-FMRP antibodies, and digested with ribonuclease T1. The washed pellets were suspended in 40 μl SDS loading buffer containing no reducing agents, heated for 10 min at 70° C., applied to 12% SDS/PA gels, and transferred to a 0.45 um nitrocellulose membrane in Towbin buffer. Western blot analysis of a duplicate membrane indicated that FMRP migrated at 80 kDa. A band encompassing approximately the 75-85 kDa molecular weight range was excised, transferred to TRI-Reagent, and vortexed vigorously for 15 min at 37° C. RNA was isolated and analyzed by RTqPCR.

SN were cross-linked with ultraviolet light (UVL), immunoprecipitated with anti-FMRP antibodies, digested with T1 ribonuclease and analyzed by SDS-PAGE. FMRP immunoreactive material (80 kDa) was excised and analyzed by real time quantitative PCR (RTqPCR). The amplicon encompassing the G-rich sequence of APP mRNA gave a positive signal that was approximately five-fold greater than that of the predicted G-quartet motif-containing sequence immediately downstream. These results demonstrated that APP mRNA was a target for FMRP binding, presumably via the coding region putative G-quartet.

These experiments also illustrated that FMRP directly binds to APP mRNA, suggesting that FMRP regulates APP mRNA expression in the brain. Modulation of FMRP binding would permit APP translation to be regulated and could control subsequent APP protein and proteolytic product accumulation (including Aβ).

### Example 2

### Activation of Glutamate Receptor Increases APP Protein Production

To determine whether modulation of mGluR affected APP translation and protein production, SN and neuronal cells were contacted as described below with DHPG, an agonist of group 1 mGluRs. The mechanism underlying FMRP-mediated translational repression is controversial (Bear et al., 2004 Trends Neurosci 27, 370-377), and alterations in the association of FMRP with polyribosomes, small nontranslated RNAs or other proteins have all been proposed (Feng et al., 1997 Mol Cell 1, 109-118; Zalfa et al., 2003 Cell 112, 317-327; Bagni et al., 2005 Nat Rev Neurosci 6, 376-387; Ceman et al., 1999 Mol Cell Biol 19, 7925-7932).

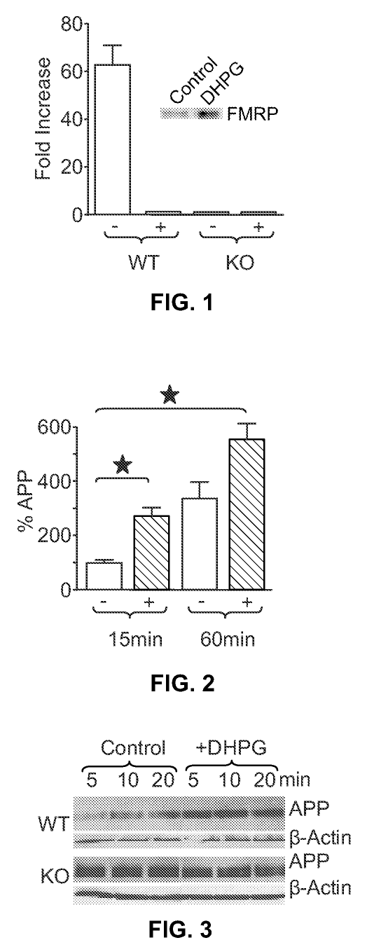

SN were isolated from normal mouse brain and then treated with the group 1 mGluR agonist DHPG. Immunoprecipitation studies were performed to determine if FMRP associated with APP mRNA. FMRP-associated APP mRNA was detected in untreated controls, whereas treatment of WT SN with DHPG resulted in dissociation of FMRP from APP mRNA. FMRP was immunoprecipitated from WT SN (60 min after DHPG treatment) and the pellet reverse-transcribed and analyzed by RTqPCR. APP mRNA was readily detected in anti-FMRP IP pellets in untreated WT SN (FIG. 1). However in SN from FMRP knockout (KO) mice (which have constitutively elevated APP), APP mRNA associated with FMRP could not be detected after up to 40 cycles of RTqPCR with or without DHPG treatment. Duplicate immunoprecipitations (IPs) in the absence of 7G1-1 FMRP antibody failed to detect APP mRNA within 40 cycles of PCR.

The >60-fold difference in FMRP-associated APP mRNA was highly significant. Evaluation at earlier times after DHPG treatment revealed that the APP mRNA-FMRP complex was lost within 5 min of contacting SN with DHPG. IP of FMRP from WT SN followed by western blot analysis (FIG. 1, inset) or ³⁵S-methionine (⁵S-Met) incorporation analysis demonstrated that DHPG treatment did not interfere with the ability of anti-7G1-1 antibody to bind to FMRP. Both experiments showed that significant amounts of FMRP immunoprecipitated from the DHPG-treated WT SN.

These data suggested a physical interaction between FMRP and APP mRNA that was responsible for APP translational repression, and that mGluR₅ activation rapidly moderated repression by affecting FMRP-APP mRNA binding. Loss of FMRP/APP mRNA interaction resulted in rapid, pulsatile protein expression in dendrites. The role of group 1 mGluR₅ in modulating APP translation repression suggested that inappropriate or dysfunctional APP translation could be treated by inhibiting mGluR₅ activity.

### Example 3

### FMRP Regulated Synaptic Synthesis of APP and Regulated Dendritic APP Levels in Cultured Neurons

To further assess FMRP regulation of APP levels in brain, cortical SN were prepared from WT mice and overall protein synthesis analyzed in response to DHPG (100 μM)-induced mGluR₅ activation. SN were metabolically active as assessed by ³⁵S-Met incorporation. To assess de novo APP synthesis, ³⁵S-labeled SN were immunoprecipitated with anti-APP antibodies. After 15 min of incubation, untreated SN were found to translate only modest amounts of APP; however, APP translation rapidly increased by (2.7-fold) following DHPG treatment. After 1 hr, APP remained elevated in stimulated SN over control, but the difference was less (1.6 fold) than at 15 min, suggesting more persistent translation in the unstimulated controls, slowing of new synthesis after stimulation and/or compensatory protein turnover in the DHPG-treated samples (FIG. 2).

In order to assess changes in steady-state APP levels, rather than new protein synthesis, APP was measured in WT and Fmr-1 KO SN in response to DHPG by western blot analysis (FIG. 3). Steady state levels of APP were substantially higher in KO SN compared to WT, and remained constant in KO SN irrespective of DHPG treatment. In contrast, there was a rapid increase in total APP levels within 5 min of DHPG treatment (1.6-fold, n=3) in WT SN, which was completely absent in KO SN. Within 20 min of DHPG, APP levels in WT SN approached those seen in unstimulated KO SN (FIG. 3). Irrespective of treatment, β-actin levels remained constant in both WT and KO SN. Protease inhibitors increased steady state levels of APP in WT SN to those seen in KO SN.

These data suggested that APP mRNA was translationally repressed by FMRP in unstimulated WT SN. mGluR₅ activation rapidly de-repressed APP production as shown for FMRP and PSD95 (Todd et al., 2003 Proc Natl Acad Sci USA 100, 14374-14378; Greenough et al., 2001 Proc Natl Acad Sci USA 98, 7101-7106). APP levels during maximal de-repression approached those seen constitutively in Fmr-1 KO cells. After cessation of mGluR₅ signaling, APP levels dropped presumably due to degradation (which appeared more robust in WT than KO cells). These results were consistent with the conclusion that FMRP inhibited translation of APP mRNA, and that stimulation of group 1 mGluR₅ signaling using a mGluR₅ receptor agonist permitted increased expression of APP mRNA.

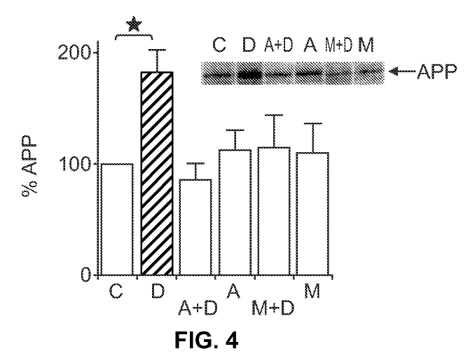

These conclusions were tested by assessing the effects on APP translation of contacting SN with a group 1 mGluR₅ antagonist, 2-methyl-6-(phenylethynyl)-pyridine (MPEP). MPEP blocked DHPG-induced stimulation of APP translation. To assess the effect of MPEP, a specific mGluR₅ antagonist, on DHPG stimulation of de novo APP synthesis, SN were pre-treated with 10 μM MPEP for 10 min prior to the addition of ³⁵S-Met for 5 min and stimulation with 100 μM DHPG for 15 min. ³⁵S-Met-labeled APP was immunoprecipitated with anti-APP antibodies and analyzed by SDS-PAGE. The mGluR₅-specific inhibitor MPEP blocked DHPG-mediated synthesis of APP to the same extent as the non-specific translational inhibitor anisomycin (40 μM) (FIG. 4).

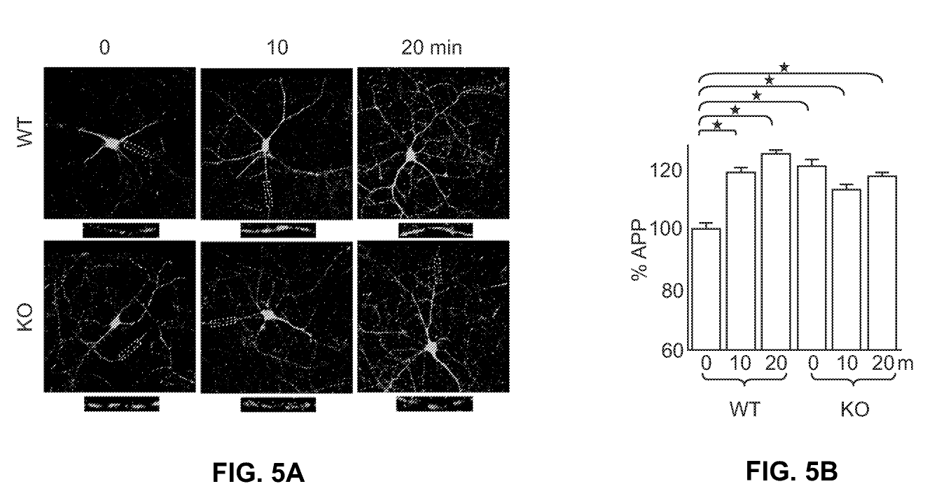

It was recognized that SN are a relatively crude preparation of pre- and post-synaptic dendrites that are contaminated with other cell types, such as astrocytes, which form synapses with neurons. Thus, primary E18 cortical neuron cultures were prepared from WT and Fmr-1 KO brains and dendritic APP levels assessed using an immunofluorescence assay. APP was found in the cell body as well as dendritic puncta of both WT and Fmr-1 KO neurons (as shown in FIG. 5A). There was a 21% increase in the basal level of APP in untreated dendrites from Fmr-1 KO neurons compared to WT (FIG. 5B). Neurons stimulated with DHPG for 10 and 20 min. prior to cell fixation showed an 18-25% increase in dendritic APP levels in WT but no increase in Fmr-1 KO cultures (FIG. 5B).

These data confirmed findings in SN that (1) Fmr-1 KO mice had higher basal synaptic levels of APP and (2) DHPG increased APP levels selectively in WT samples. These data also demonstrated that FMRP and mGluR₅ activation regulated APP synthesis in both FVB and C57BL/6 mice, as the SN used in the experiments set forth above were prepared from the former and the primary cortical neurons from the latter mouse strain. No differences in APP levels were observed in cell bodies of WT versus KO samples with or without DHPG treatment. Therefore, the expected reduction in APP after mGluR₅ inhibition was limited to dendrites, which constitute 98% of the surface area of neurons (Kandel et al., 2000, Principles of Neural Science, 4^{th} ed. McGraw-Hill, New York).

The studies above demonstrated increased APP in fmr-1^{-/-} synaptoneurosomes prepared from young mice (P14-17), as well as increased dendritic APP in fmr-1^{-/-} primary neuronal cells compared to WT controls. To assess the impact of MPEP on APP expression in primary neuronal cells, primary neuronal cells were prepared from WT C57BL/6 embryos (age: E17/18) were treated with MPEP and analyzed for APP expression by confocal microscopy. APP was localized in dendritic puncta and levels were quantitated as average particle intensity.

Specifically, primary mouse neuronal cells were prepared from 10 embryos (age E17/18) dissected from a timed pregnant C57BL/6 female as previously described and cultured for 15 days. Cells were treated with 10 μM MPEP for the indicated durations prior to fixation and staining with anti-22C₁₁ against the amino-terminus of APP (Chemicon mAB348; 1:2000 for 1 hr) and visualized with goat anti-mouse rhodamine-conjugated secondary antibody (Invitrogen; 1:500 for 30 min in the dark). Staining of dendritic puncta was analyzed by confocal microscopy and quantitated with Image J software using the Analyze Particles function (a minimum of 475 particles were analyzed per treatment) (Rasband, W. S., Image J, U.S. National Institutes of Health, http://rsb.info.nih.gov/ij; 1997-2006). Duplicate slides were stained for each treatment and the number and intensity of dendritic puncta from three areas of each slide were analyzed and averaged. All MPEP-treated samples were statistically different from untreated controls by t-test analyses (p<0.005) and expressed as standard error of the mean (SEM).

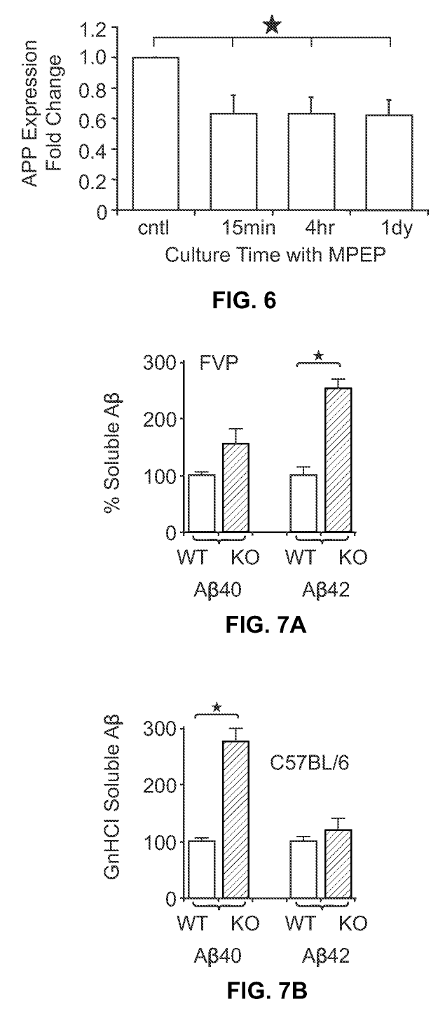

A 37% decrease in dendritic APP after 15 min MPEP treatment, which persisted at 4 hr and 1 day (statistically significant at all time points, p<0.005) (FIG. 6). The area per particle was also assessed, which decreased at 15 min, 4 hr and 1 day, as well as the number of particles per area, which increased. Hence, both the average particle intensity and the area of each particle decreased, by 37% and 22%, respectively, while and number of particles in a defined area increased by 29%. Overall APP levels in dendritic puncta decreased despite two opposing phenomena in which the size of the stained puncta decreased but the number of puncta increased. In summary, MPEP decreased APP expression in primary neuronal cells.

In summary, these studies demonstrated that FMRP regulated synaptic synthesis of APP and dendritic APP levels in cultured neurons and that MPEP blocks APP translation in cultured neurons.

### Example 4

### Soluble Aβ₁₋₄₀ or Aβ₁₋₄₂ Were Increased in Fmr-1 KO Mouse Brain and in Human FXS Brain

Increased translation of APP provides more targets for cleavage by β- and γ-secretases. Hence, in view of the results set forth above, Fmr-1 KO mice were expected to have increased A13 production with aging. To assess the validity of this expectation, right brain hemispheres from the WT and KO middle-aged FVB mice (11-13 months old) were homogenized in protein extraction buffer containing 1% Triton X-100 and protease inhibitors, and the soluble material was analyzed by ELISA for Aβ₁₋₄₀ and Aβ₁₋₄₂. The Fmr-1 KO mouse brain contained 1.6-fold more Aβ₁₋₄₀ and 2.5-fold more Aβ₁₋₄₂ than WT controls (FIG. 7A).

Aβ_{1-40/1-42} levels were also tested in C57BL/6 mice (12-14 months old) to ensure that the results set forth above were not FVB strain-specific. An increase in soluble Aβ₁₋₄₀ or Aβ₁₋₄₂ levels was not observed in Fmr-1 KO C57BL/6 brain samples, but guanidine-soluble fractions showed a 2.8-fold increase in Aβ₁₋₄₀ and a 1.2-fold increase in Aβ₁₋₄₂ (FIG. 7B). Thus, brains of two distinct murine strains lacking Fmr-1 showed increased pathogenic Aβ species over time, consistent with the role discerned from the experiments disclosed herein of FMRP in repressing APP translation.

Human FXS brain and serum samples were examined for the presence of Aβ species. The role of APP in the pathology of Alzheimer's disease has been extensively studied, but there is very limited data regarding APP mRNA and protein levels in humans with FXS. One group (D'Agata et al., 2002, Neurobiol Dis 10, 211-18) showed elevated APP mRNA in the cerebral cortex, hippocampus and cerebellar cortex in fmr-1 KO mice, but these differences have not been observed in cortical synaptoneurosomes (Westmark et al., 2007, PLoS Biol 5, e52). Aβ1-40 and Aβ1-42 as well as APP were increased in whole brain lysates and cultured neurons, respectively, of fmr-1 KO mice (Westmark et al., 2007, PLoS Biol 5, e52)).

Despite the prevalence of FXS, robust biomarkers for disease detection or severity have not been described. APP/APPβ and Aβ levels were examined in the blood of full-mutation FXS males in order to establish the utility of these proteins as FXS biomarkers. Blood was drawn from donors into lithium heparin (Li)-coated blood collection tubes and spun at 1500 rpm. The plasma supernatant was removed and frozen at −80° C. Anti-coagulated blood was handled so that peripheral blood mononuclear cells (PBMC) were isolated within 24 hr of blood draw. Plasma was thawed and clarified at 12,000 rpm for 10 min at 4° C. prior to ELISA assays for APP/APPα, Aβ₁₋₄₀ and Aβ₁₋₄₂ per the manufacturer's instructions (BioSource, Int., catalog #KHB0051, KHB3482, KHB3442) with the following modifications for the Aβ assays: (1) the sample volume was doubled from 50 μL to 100 μL, (2) the incubation time was extended from 3 hr to overnight at 4° C., and (3) after the overnight incubation, the samples were removed from the antibody-coated wells prior to addition of the detection antibody. PBMC were isolated as previously described (Westmark et al., 2001, Brain Res Mol Brain Res. 90, 193-201) and cultured for 24 hr prior to harvesting the cells and culture media for ELISA analyses. Soluble Aβ₁₋₄₀ in hippocampus and neocortex of control and FXS brain autopsy tissue was assessed by ELISA after preparation of detergent-soluble lysates as previously described (Westmark et al., 2007, PLoS Biol 5, e52, incorporated by reference in its entirety herein). Total Aβ was extracted from brain tissue by homogenization in 8M GnHCl/50 mM Tris (pH 7.5) buffer followed by gentle mixing overnight.

A significant two-fold decrease in plasma Aβ₁₋₄₂ was detected for individuals with FXS compared to age-matched controls. Plasma Aβ₁₋₄₀ and amyloid precursor protein (APP), the parental molecule from which Aβ is derived were unchanged. Autopsy brain tissue showed trends for increased APP and Aβ levels in FXS hippocampus and neocortex compared to controls.

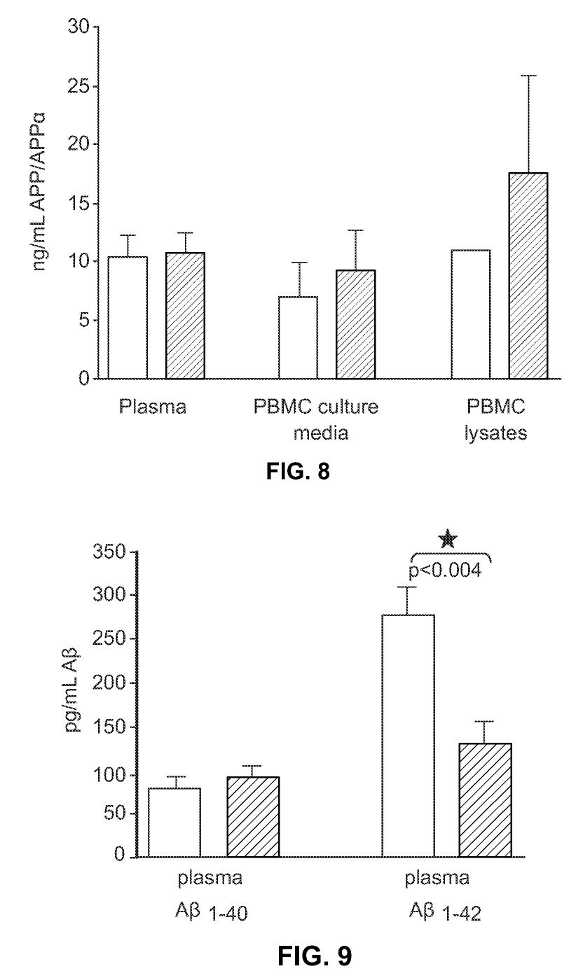

Measurement of APP/APPβ in human samples, revealed that APP/APPα levels were comparable in FXS plasma (mean age 21.3; SD 7.4; age range 9-32) and control plasma (FIG. 8). These results are distinct from those seen in two autistic FXS children (ages 6 and 14) (Sokol et al., 2006, J. Child Neurol. 21, 444-449) suggesting that APP expression and processing decrease with age. Indeed, APPα levels are lower in children 10 years and older than for children 7 years and younger (Sokol et al., 2006, J. Child Neurol. 21, 444-449). While Aβ₁₋₄₀ levels were also unchanged between FXS and controls, Aβ₁₋₄₂ in blood plasma was significantly lower in the FXS group (2.1-fold decrease, p<0.004) (FIG. 9). APP/APPα levels in PBMC samples from FXS and control donors were also assessed. There was not a statistically-significant increase observed in cell-associated APP or secreted APPα (FIG. 8). APP_{695/751/770} mRNA levels in PBMC measured by RTqPCR were not statistically different between control and FXS samples.

The lower levels of Aβ₁₋₄₂ in blood plasma initially appeared contradictory to previous results wherein higher levels of Aβ and APP were observed in fmr-1 KO mouse brain (Westmark et al., 2007, PLoS Biol 5, e52). Despite the increased levels of Aβ in the brain and well-established roles for APP and Aβ in AD progression, blood and brain levels of Aβ did not always correlate (Freeman et al., 2007, J Neuropathol Exp Neurol 66, 264-271; Hansson et al., 2008, Neurobiol Aging (epub ahead of print). A reduced Aβ₁₋₄₂/Aβ₁₋₄₀ ratio is a risk factor for AD (Graff-Radford et al., 2007, Arch Neurol. 64, 354-362; Sobow et al., 2005, Acta Neurobiol Exp (Wars), 65, 117-124; van Oijen et al, 2006, Lancet Neurol. 5, 655-660), but the absolute plasma level of Aβ in mild cognitive impairment (MCI) may be reduced, normal or elevated despite CNS accumulation (Hansson et al., 2008, Neurobiol Aging (epub ahead of print); Mehta et al., 2000, Arch Neurol. 57, 100-105; Ringman et al., 2008, Neurology 71, 85-92; van Oijen et al., 2006, Lancet Neurol 5, 655-660). Plasma Aβ₁₋₄₂ levels are increased in women with mild cognitive impairment (preclinical stage), but drop to control levels by the time of a probable AD diagnosis (Assini et al., 2004, Neurology 63, 828-831). Similar results were seen in 1,125 elderly persons without dementia where higher plasma Aβ₁₋₄₂ levels at the onset of the study were associated with a 3-fold increase in AD risk while decreased plasma Aβ₁₋₄₂ and a lower Aβ₁₋₄₂/Aβ₁₋₄₀ ratio accompanied conversion to AD (Schupf et al., 2008, Proc Natl Acad Sci USA 105, 14052-14057). In Trisomy 21 (Down's syndrome), elevated plasma Aβ₁₋₄₂ is associated with earlier onset of AD (Schupf et al., 2007, Arch Neurol 64, 1007-1013) and the Aβ₁₋₄₂/Aβ₁₋₄₀ blood plasma ratio is lower than in controls (Mehta et al., 2007, J Neuro Sci 254, 22-27). Thus, the results set forth herein of reduced Aβ₁₋₄₂/Aβ₁₋₄₀ ratio (1.4:1) compared to control plasma (3.4:1) is consistent with other amyloidogenic diseases and suggests that the brain acts as a sink for Aβ and that lower blood plasma levels may indicate increased brain deposition.

Previously reported control plasma levels of Aβ range from 130-208 pg/mL for Aβ₁₋₄₀ and 15-60 pg/mL for Aβ₁₋₄₂ (Hansson et al., 2008 (epub ahead of print); Mayeux et al., 2003; van Oijen et al., 2006). Thus, Aβ₁₋₄₀ levels are in agreement with the literature, but Aβ₁₋₄₂ levels are significantly higher. Regardless of the absolute levels of Aβ₁₋₄₂ in plasma, which may vary based on plasma preparation, assessment technique employed or ELISA antibodies used for detection, a statistically significant decrease in Aβ₁₋₄₂ in blood plasma collected from FXS patients was measured.

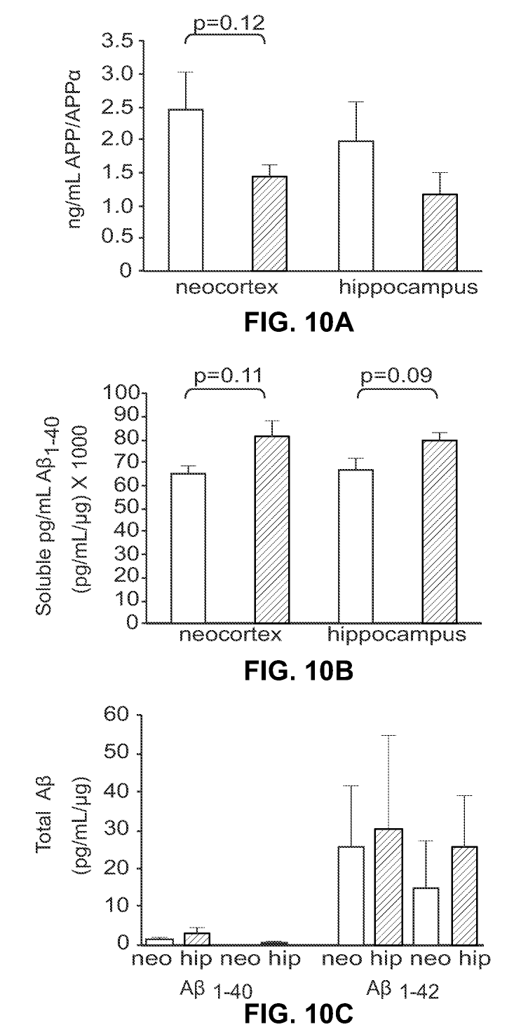

APP/APPα, Aβ₁₋₄₀ and Aβ₁₋₄₂ in hippocampal and neocortical (pre and post central gyri) samples of left cerebral hemisphere from control and FXS brain autopsy tissue were measured. All of the donors were Caucasian males and their ages ranged from 21-85 years old. Controls were gender- and age-matched by the University of Maryland Brain Bank. Sample size was limited due to the availability of human tissue. The data represents analyses of four FXS and three control brains. A strong trend toward increased neocortical and hippocampal Aβ₁₋₄₀ in soluble lysates prepared by detergent extraction of the FXS brain samples was observed (FIG. 10B) while APP/APPα levels are decreased (FIG. 10A). Despite the small sample size, the 1.7-fold decrease in APP/APPα in neocortex is nearly statistically significant (p=0.12). Neocortical and hippocampal lysates were prepared by guanidine extraction and likewise showed a strong trend for increased Aβ₁₋₄₀ and Aβ₁₋₄₂ levels in the FXS samples (FIG. 10C). These results with human FXS autopsy brain tissue were similar to fmr-1 KO mouse data, which demonstrated elevated Aβ in the brain. In summary, these data illustrated that Aβ₁₋₄₂ is a plasma-based biomarker for FXS and that therapies directed at reducing CNS Aβ as used to treat Alzheimer's disease may be applicable to FXS.

Drug efficacy in FXS is currently assessed by behavioral testing methods alone because there is a dearth of reliable blood biomarkers for FXS. Other studies have demonstrated reduced cAMP production in platelets (Berry-Kravis et al., 1993, Am J Med Genet. 45, 81-87) and delayed early-phase phosphorylation of extracellular-signal regulated kinase (ERK) in lymphocytes (Weng et al., 2008, Am J Med Genet B Neuropsychiatr Genet. 147B, 1253-1257) from FXS patients. The experimental data described herein illustrated a significant two-fold decrease in Aβ₁₋₄₂ in FXS blood plasma. These results in conjunction with APP ELISA data from autistic children (Bailey et al., 2008, Int J Clin Exp Med 1, 338-344; Sokol et al., 2006, Child Neurol 21, 444-449), suggested that both APP and Aβ can be useful biomarkers for FXS. A blood test for FXS biomarkers would enable clinicians to correlate drug efficacy with biologic outcome measures as well as with improvements in behavioral and cognitive function.

### Example 5

### Reduced mGluR₅ Activation and Reduced Synthesis of APP and Accumulation of Aβ Upon Administration of mGluR₅ Antagonist

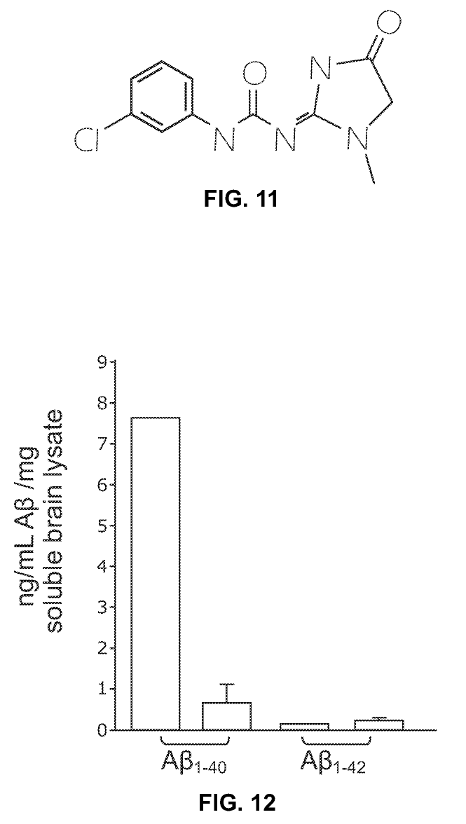

To determine the impact of an mGluR₅ antagonist on mGluR₅ activation and hence synthesis of APP and accumulation of Aβ in the brain, fenobam (FIG. 11) was administered to mice. In initial experiments, Tg2576 mice were utilized, which are an Alzheimer disease (AD) mouse model that over-express the coding region of human APP containing the Swedish familial AD mutation (hAPP_{Swe}). These mice exhibit a basal 5-fold increase in Aβ₁₋₄₀ and a 14-fold increase in Aβ₁₋₄₂ expression (Hsiao et al., 1996, Science 274: 99-102).

Mice were fed a fenobam-supplemented mouse food that was a purified ingredient diet formulated by Research Diets, Inc. (New Brunswick, N.J.) to match Purina 5015 lab diet in protein, carbohydrate and fat calories. For chronic dosing, the diet was supplemented with 0.2 g fenobam per kg lab diet and colored red for visual differentiation with the placebo food. No adverse side effects from the drug were observed, and notably, no incidences of premature death occurred. Tg2576 have exhibited an increased seizure rate and premature death rate of 40% by 60 days of age (Westmark et al., 2007, Int J of Clin and Exp Path 1: 157-168). In the Tg2576 cohort on control food, 2 of the 4 mice died during the treatment period. Tg2576 mice on food supplemented with fenobam as described above exhibited normal weight gain over a one-month period as well as typical home cage behavior and activity.

After one month of chronic fenobam dosing, brain tissue was harvested and Aβ levels measured. Specifically assessed by ELISA were human (expressed from the APP_{Swe} transgene) and mouse (endogenous) Aβ₁₋₄₀ and Aβ₁₋₄₂ levels. No change in human Aβ levels between control and fenobam-fed mice was observed; however, a highly significant 11.5-fold decrease in mouse Aβ₁₋₄₀ levels with fenobam treatment in the male Tg2576 was observed (FIG. 9). The level of Aβ₁₋₄₀ in the one male Tg2576 mouse on control food (7.632 ng/mL/μg) was comparable to the Tg2576 female mouse on control food (7,408 ng/mL/μg) and to three Fmr-1 KO female mice on control food (8,650 ng/mL/μg; sem=2179) during the same treatment period. Thus, the reduction (to 60 ng/mL/μg) in Aβ₁₋₄₀ found in the Tg2576 males on fenobam food was highly significant. There was no change in mouse Aβ₁₋₄₂ levels with fenobam treatment, albeit endogenous Aβ₁₋₄₂ was present at very low concentrations in both treated and untreated mice.

The absence of a fenobam effect on the human Aβ levels is likely due to the nature of the hAPP_{Swe} transgene and sheds additional light on the nature of FMRP repression of APP translation. The mouse genome of Tg2576 mice contains only the coding region of hAPP_{Swe} as a transgene. FMRP is known to bind to a G-rich region in the coding region of APP mRNA and this binding is abrogated with group 1 mGluR₅ activation; however, FMRP also binds to the 3′-UTR of APP mRNA. While it is unclear if FMRP binds directly to the 3′-UTR or as part of an mRNP complex, the results obtained in these experiments from the transgene that lacks the 3′-UTR of APP mRNA indicated that regions outside of the coding region were important for FMRP repression of APP translation. In contrast with the results obtained with the human transgene, the endogenous mouse gene putatively containing all of the FMRP regulatory elements displayed a profound decrease in Aβ₁₋₄₀ levels in response to mGluR₅ blockade.

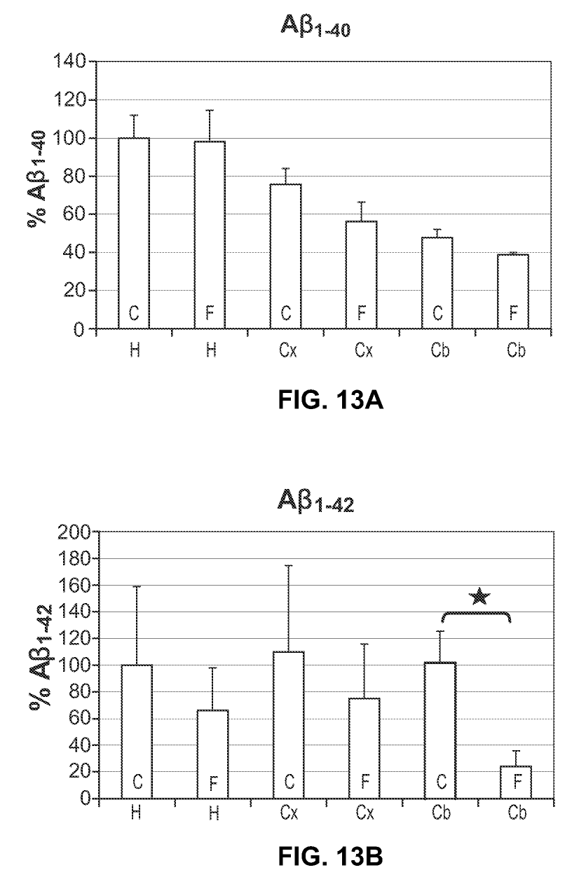

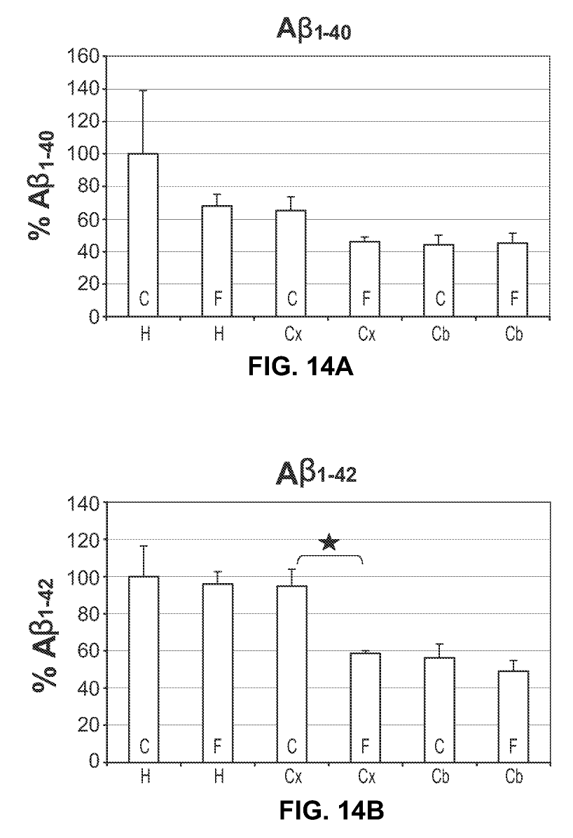

The initial chronic dosing experiment with Tg2576 mice (FIG. 12) commenced shortly after the fenobam food was synthesized and showed a significant decrease in Aβ₁₋₄₀ in a small cohort of animals. The efficacy of fenobam on Aβ reduction in Fmr-1 KO mice was examined in two subsequent studies, which gave more modest, but statistically significant, reductions in Aβ₁₋₄₂ in either the cerebellum (FIG. 13) or the cortex (FIG. 14). The results shown in FIG. 13 were obtained from Fmr-1 KO mice treated with either placebo or fenobam food (3 mice per cohort) for 2 months prior to harvesting brains and assessing mouse Aβ₁₋₄₀ and Aβ₁₋₄₂ levels by ELISA. There was a 4.2-fold reduction in Aβ₁₋₄₂ in the cerebellum in response to fenobam, which was statistically significant (Student t-test, p<0.05), and a 26% and 19% decrease in Aβ₁₋₄₀ in cortex and cerebellum, respectively. There were also modest reductions in Aβ₁₋₄₂ in hippocampus and cortex.

To assess the consistency of these results, Fmr-1 KO mice (3 mice per group) were treated with placebo versus fenobam food for 1 month (these results are shown in FIG. 14). A 40% decrease in Aβ₁₋₄₂ in the cortex was observed and was statistically significant (Student t-test, p<0.02). While the brain region affected was different in the results shown in FIGS. 13 & 14 (cerebellum versus cortex), both studies showed a modest but significant reduction in Aβ₁₋₄₂ in Fmr-1 KO mouse brain.

The consistent results of the experiments set forth herein are that modest to profound reductions in Aβ were observed after fenobam treatment (a specific mGluR₅ antagonist) in three separate experiments with 2 different strains of mice.

### Example 6

### APP Transgenic Mice Had a Lower Threshold for Seizures and Treatment with mGluR₅ Antagonist Ameliorates AD Phenotypes

To better study the phenotypes associated with AD and FXS such as cognitive decline, seizures and dendrite dysmorphogenesis, a new mouse model with exacerbated APP/Aβ expression was generated. Frm-1 KO (Fragile X mice) were crossed with Tg2576 (AD mouse), which generated a new FRAXAD mouse model. FRAXAD mice exhibited 23% more A140 than Tg2576 in whole brain lysates (14-16 day old mice), and had an increased juvenile mortality rate. Both Tg2576 and FRAXAD had average seizure scores greater than 4 on a 5-point scale compared to 2.42±0.31 for WT male C57BL/6 mice in response to the chemical convulsant pentylenetetrazole (PTZ), which characteristic of these mice was used in the assays set forth below.

To assess the impact of mGluR₅ inhibition on seizure phenotypes, Frm-1 KO, Tg2576, and FRAXAD mice were treated with mGluR₅ antagonist, MPEP. Tg2576, FRAXAD and littermate controls were treated with 30 mg/kg MPEP (via I.P. injections) 30 min prior to seizure induction with PTZ. The experimental results presented in Table 2 support MPEP's effect as an anti-convulsant in both WT and Fmr-1 KO mice. WT male mice reach a grade 3 seizure, fully developed minimal seizure with clonus of the head muscles and forelimbs, at a rate of 50% in response to 50 mg/kg PTZ, but only 20% achieve a grade 3 seizure with MPEP treatment 30 min prior to PTZ (statistically significant, χ² test, p<0.0005). Likewise, there was a reduction from 11% to 0%, 46% to 25%, and 40% to 33% in WT females, Fmr-1 KO males and Fmr-1 KO females, respectively when tested using the seizure induction assay.

TABLE 2

| | | | | | MPEP + |
|---|---|---|---|---|---|
| | | PTZ | MPEP + PTZ | PTZ | PTZ |
| | Gender | % grade 3 | % grade 3 | % death | % death |
| WT | M | 50 | 20* | 0 | 0 |
| | F | 11 | 0* | 0 | 0 |
| FMR-1 KO | M | 46 | 25* | 0 | 0 |
| | F | 40 | 33 | 0 | 0 |
| Tg2576 | M | 100 | 100 | 71 | 75 |
| | F | 100 | 75 | 83 | 50* |
| FRAXAD | M | 83 | 80 | 42 | 60* |
| | F | 100 | 100 | 86 | 40* |

*χ² test was used to compare seizure incidence and death ±MPEP, p < 0.0005.

MPEP reduced the grade 3-seizure rate in Tg2576 females from 100% to 75%, but there was no reduction in the seizure rate for Tg2576 males or FRAXAD mice. The lower death rates in Tg2576 and FRAXAD females after MPEP likely resulted from the anti-anxiety activity of the drug, whereas the rates in males likely resulted from the regulatory mechanism of hAPP or hAp production in these mouse lines (Tg2576 or FRAXAD). The Tg2576 and FRAXAD mice expressed only the coding region of hAPP_{Swe}, which lacks regulatory UTR elements and appears non-responsive to mGluR₅ blockade.

APP transgenic mice exhibited a lower threshold for audiogenic-induced seizures. Home cage seizure activity was observed for two AD mouse strains that over-expressed hAPP/Aβ (Tg2576 and FRAXAD). Tg2576 mice over-expressed hAPP_{Swe} and hAβ. FRAXAD were a cross between fmr-1^{-/-} (Fragile X mouse model) and Tg2576 (AD mouse model) and generated 23% more brain hAβ₁₋₄₀ than Tg2576 (Westmark et al., 2008, Int J Clin Exp Pathol 1: 157-168). Fmr-1^{-/-} mice, which over-expressed mouse APP and Aβ, had a high sensitivity to audiogenic stimulation. It was determined whether hAPP_{Swe}/hAβ over-expression in an fmr-1 null background (FRAXAD mice) increased AGS. C57BL/6 fmr-1^{-/-} mice were sensitive to AGS from 15-47 days with a peak at 21 days (Yan et al., 2005, Neuropharmacology 49: 1053-1066). AGS in WT, fmr-1^{-/-}, Tg2576 and FRAXAD mice was examined at 21 days of age. Mice were transferred to a Plexiglas box (13″L×8″W×7″H) shortly after weaning and exposed to high pitch siren (118 dB) from a personal body alarm (LOUD KEY™).

TABLE 3

| | | | % wild | % | |
|---|---|---|---|---|---|
| | gender | n | running | seizure | % death |
| WT | M | 22 | 12 | 6 | 6 |
| | F | 17 | 5 | 5 | 5 |
| Fmr-1 KO | M | 8 | 63 | 50 | 38 |
| | F | 8 | 50 | 38 | 38 |
| Tg2576 | M | 5 | 40 | 40 | 40 |
| | F | 11 | 64 | 36 | 36 |
| FRAXAD | M | 13 | 100 | 92 | 62 |
| | F | 8 | 73 | 64 | 55 |
| dKO | M | 29 | 100 | 97 | 66 |
| | F | 13 | 100 | 100 | 62 |
| APP Het | M | 13 | 8 | 0 | 0 |
| | F | 18 | 11 | 0 | 0 |
| APP KO | M | 4 | 25 | 25 | 0 |
| | F | 6 | 50 | 33 | 33 |
| APP Het, | M | 13 | 62 | 23 | 23 |
| Fmr-1 KO | F | 10 | 40 | 30 | 20 |

*χ² test was used to compare seizure incidence in the Fmr-1 KO and APP Het/Fmr-1 KO male group

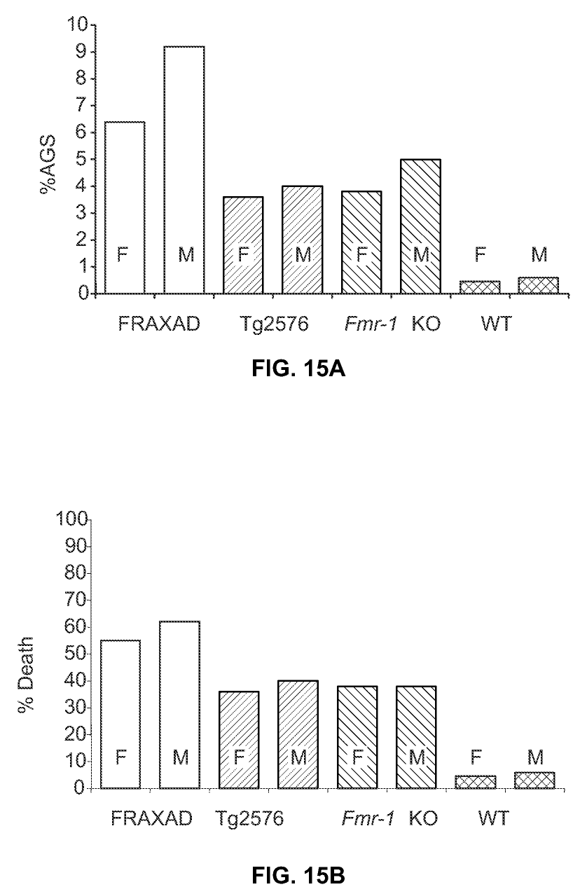

The number of mice exhibiting wild running (WR), tonic seizures (AGS) and respiratory arrest/death (Table 3 & FIGS. 15A, 15B) were scored. Inbred C57BL/6 are resistant to AGS (Schlesinger et al., 1970, In: Anonymous pp. 219-219-257), thus only a 5 and 6% AGS rate for WT females and males, respectively. The following AGS rates were observed: 6, 50, 40 and 92% rate of AGS in WT, fmr-1^{-/-}, Tg2576 and FRAXAD male mice, respectively, and 5, 38, 36 and 64% in the females. Thus, there is an additive effect on seizure propensity in the FRAXAD mice, which have exacerbated hAPP_{Swe}/Aβ production in an fmr-1^{-/-} background. AGS rates of 43% and 60% were observed in female and male, respectively, DS mice compared to 20% and 0% in littermate controls. It was also found that for fmr-1^{-/-} mice, 50% (females) and 63% (males) showed WR, 38% (females) and 50% (males) AGS, and 38% a death rate for both genders. These seizure rates were lower and death rates were higher than were described by others, who found approximately 78% AGS and 8% mortality in fmr-1^{-/-} male mice in the C57 background (Yan et al., 2005, Neuropharmacology 49: 1053-1066). There was variability in AGS rates for fmr-1^{-/-} KO mice in the literature (Chen et al., 2001, Neuroscience 103: 1043-1050; Yan et al., 2005, Neuropharmacology 49: 1053-1066; Dolen et al., 2007, Neuron 56: 955-962) and strain background appeared to play an important role; however, in all cases fmr-1^{-/-} mice exhibited a strong AGS phenotype whereas WT controls do not. The experimental results set forth herein, comparing AGS rates in male and female fmr-1^{-/-} mice were in agreement with the literature as both genders exhibit equal propensity for AGS (Qin et al., 2005, Neuroscience 135: 999-1009).

In Tg2576 mice, a 36-40% seizure rate was observed. Thus, these results demonstrated that a mouse model for AD was susceptible to AGS and strongly suggested that dysregulation of APP and/or Aβ contributes to AGS susceptibility. The Tg2576 mice exhibited 64% (females) and 40% (males) WR and 36% (females) and 40% (males) rates for both AGS and respiratory arrest (Table 3). For comparison, the following rates were observed for FRAXAD mice: 73% (females) and 100% (males) WR, 64% (females) and 92% (males) AGS, and 55% (females) and 62% (males) (Table 3). Thus, there was an additive effect on seizure susceptibility in the FRAXAD males (92%) compared with the parental fmr-1^{-/-} (50%) and Tg2576 (40%) strains (FIG. 15A). There was also increased AGS susceptibility in the FRAXAD females (64%) compared with 38% (fmr-1^{-/-}) and 36% (Tg2576) (FIG. 14A). Similarly, there was an increased death rate in FRAXAD mice compared to Tg2576 and fmr-1^{-/-} mice (FIG. 15B). All of the Tg2576 (males and females) as well as fmr-1^{-/-} females that seized also died, and at least two-thirds of the FRAXAD (males and females) and the fmr-1^{-/-} males died from the AGS (Table 3). Overall, in all of the FXS and AD strains tested, there was a significant increase in seizure sensitivity compared to WT controls.

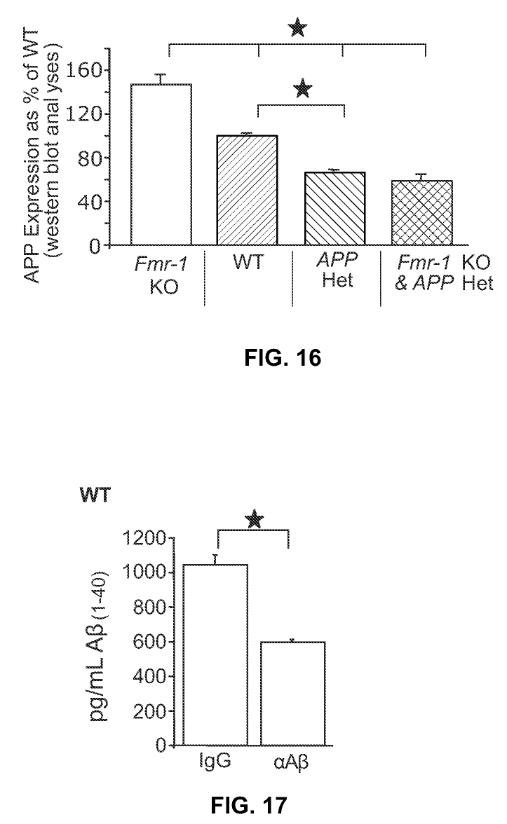

To further assess whether APP is involved in seizures, APP KO mice were crossed with Fmr-1 KO mice. The offspring of this mating were expected to revert the increased AGS phenotype found in the FRAXAD mice, but unexpectedly the exact opposite was observed. Despite the loss of APP in an Fmr-1 KO background, there was a 100% incidence rate of WR and seizures in the double KO (APP/Fmr-1) mice (Table 3, n=4). These results suggested that reduced physiological APP expression was associated with seizure activity. Thus, either too much (Tg2576 & FRAXAD mice) or too little (dKO mice) APP/sAPP/Aβ enhanced the AGS phenotype. To verify, Fmr-1^{KO} APP^{HET} mice (FRAXHAPP), which are Fragile X mice that would be expected to produce half as much mAPP as Fmr-1 KO mice, were also generated. FRAXHAPP exhibit a 0% incidence of seizures and death. Therefore, the AGS seizure phenotype observed in Fmr-1 KO and APP/Fmr-1 dKO mice can be completely reverted by genetic manipulation of APP levels. (FIG. 16).

The data above showed “genetic rescue” of AGS with FRAXHAPP mice, which were expected to have a 50% reduction in APP expression. This genetic approach was not possible in humans but very strongly suggested that APP and/or Aβ played a causative role in seizure induction. These studies demonstrated an increased incidence of seizures in mice that over-(Fmr-1 KO, Tg2576 and FRAXAD) and under-express (Fmr-1KO APPKO) APP as well as “genetic rescue” of AGS in APPHET Fmr-1KO (FRAXHAPP) mice. These data strongly supported a role for the APP, sAPP and/or Aβ in seizures and serves as an impetus to study the effect of drugs that normalize APP/sAPP/Aβ to physiological levels on seizure threshold, anxiety, spontaneous activity and memory.

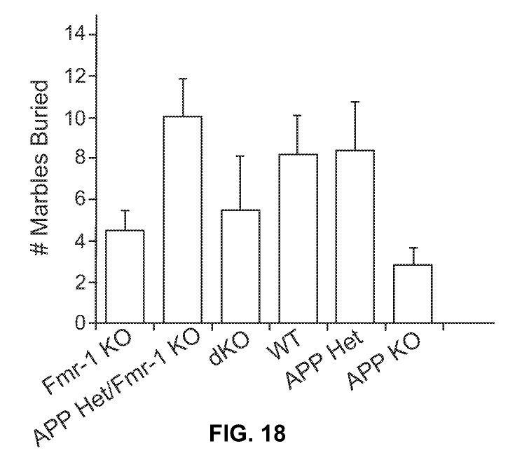

The behavior of the various APP mouse lines was tested by marble burying. Traditionally, marble burying provides an assessment of anxiety as it has been suggested that mice do not like novel objects and bury them so not to see them. Male mice were acclimated to the behavior testing room for at least 20 min before testing in the marble burying assay. Single mice were transferred to a normal housing cage lined with approximately two inches of corncob bedding. Twenty black marbles were aligned in a 4×5 grid on top of the bedding. After 30 min the mouse was removed from the cage containing marbles and the number of marbles that were half buried versus half visible were counted. WT and Fmr-1 KO mice and this difference is reverted with the Fmr-1 KO/APP Het mice (FIG. 18). The data shows that Fmr-1 KO mice bury average of 4.45 marbles, WT mice bury average of 8.22 marbles and Fmr-1 KO/APP Het mice bury average of 10 marbles. p=001 between Fmr-1 KO and Fmr-1 KO/APP Het. p=0.07 (almost statistically significant) between WT and Fmr-1 KO mice.

An alternative approach to genetic studies was passive immunization with an antibody directed against Aβ. Aβ₁₋₄₀ cortical levels were measured in adult wild-type mice that received IP injections of 50 μg nonimmune IgG or anti-APP/Aβ antibody three days prior to sacrifice. Cortical Aβ₁₋₄₀ levels as measured by ELISA and found significantly reduced for anti-APP/Aβ antibody-treated mice. (FIG. 17). The immunization was expected to induce clearance of preexisting amyloid in the brain and reverted memory deficits, without serious side effects.

TABLE 4

| Fmr-1 KO Mice | n | % WR | % AGS | % Death |
|---|---|---|---|---|
| Immunized with 12.5 μg anti- | 15 | 27 | 13 | 7 |
| APP/Aβ | | | | |
| Immunized with 12.5 μg IgG | 10 | 30 | 30 | 30 |

In conclusion, these studies illustrated that: (1) chronic dosing of Fmr-1 KO mice with fenobam, a specific mGluR₅ antagonist, as a food additive reduced Aβ₁₋₄₂ levels in cortical and cerebellar brain tissue, and (2) AD transgenic mice that over-express APP and Aβ exhibited lower thresholds to both chemically-induced and audiogenic seizures (AGS).

### Example 7

### Audiogenic Seizures in Down's Syndrome (DS) Mouse Model

Amyloid precursor protein (APP) over-expression and Aβ deposition are also strongly implicated in Down's Syndrome (DS) pathology. DS individuals carry an extra copy of the APP gene (located on chromosome 21), have up-regulated mGluR5 activity in brain, and exhibit Aβ deposition as early as age 12 and dementia by their mid-30s.

Ts65Dn mice are the most well studied and genetically complete model of DS available (Davisson et al., 1990, Prog Clin Biol Res 360: 263-280). They have three copies of most of the genes on mouse chromosome 16, the mouse correlate of human chromosome 21, including the APP gene (Reeves et al., 1995, Nat Genet. 11: 177-184). These mice over-express APP and display impaired performance on motor, spontaneous locomotion, anxiety and spatial and contextual learning and memory tests (Holtzman et al., 1996, Proc Natl Acad Sci USA 93: 13333-13338; Reeves et al., 1995, Nat Genet. 11: 177-184; Coussons-Read et al., 1996, Behav Genet. 26: 7-13; Moran et al., 2002, Physiol Behav 77: 403-409; Demas et al., 1998, Behav Brain Res 90: 199-201; Demas et al., 1996, Behav Brain Res 82: 85-92; Fernandez et al., 2008, Behav Brain Res 188: 233-237; Hyde et al., 2001, Behav Brain Res 118: 53-60; Hunter et al., 2003, Neurosci Res 45: 437-445).The WT and Ts65Dn mice are in a mixed background (mothers: B6EiC3Sn a/A-Ts(17¹⁶)65Dn females; fathers: B6EiC3Sn (C57BL/6JEi×C3H/HeSnJ) F1 males). The non-trisomic WT controls exhibited an increased propensity for AGS compared to the C57BL/6 WT mice. AGS susceptibility in Ts65Dn mice is shown in Table 4. Trisomic mice displayed a 69% WR and 54% seizure and death rates compared to 40% WR and 10% seizure and death rates for the littermate controls. In addition, treatment with fenobaum or immunization with anti-APP/Aβ antibody attenuates seizures.

TABLE 5
Audiogenic Seizure, and Anxiety Rates in Down's Syndrome
Mouse Model

| Genotype | n | % WR | % AGS | % Death |
|---|---|---|---|---|
| DS | 16 | 75 | 56 | 50 |
| DS immunized | 6 | 33 | 0 | 0 |
| anti-APP/Ab | | | | |
| DS Purina + Fenobaum | 12 | 58 | 0 | 0 |
| WT | 13 | 31 | 15 | 15 |
| WT immunized | 5 | 0 | 0 | 0 |
| anti-APP/Ab | | | | |
| WT Purina + Fenobaum | 11 | 0 | 0 | 0 |

These studies strongly support the use of mGluR5 antagonists in human clinical trials for DS as well as other neurological disorders involving the over-production of APP or one it proteolytic products.

### Example 8

### MPEP Blocks Audiogenic Seizures in FXS and AD Mouse Models

A common denominator between Fragile X Syndrome (FXS), Downs Syndrome (DS), and Alzheimer's Disease (AD) is the over-expression of amyloid precursor protein (APP) and/or beta-amyloid (Aβ). Over-expression of APP/Aβ contributes to the AGS phenotype in fmr-1^{-/-} mice and this suggested that AD and DS mouse models would exhibit reduced seizure thresholds as well. Additionally, methods that decreased APP/Aβ levels were expected to reduce AGS. Accordingly, AGS rates were examined in AD, FXS and DS mouse models. AGS rates for FRAXAD, Tg2576, and fmr-1^{-/-}, Ts65Dn were described above in Example 6. Here, the ability of 2-methyl-6-(phenylethynyl)pyridine hydrochloride (MPEP) to attenuate AGS was assessed. Tg2576 and FRAXAD mice were treated with MPEP for 30 min prior to AGS stimulation to assess the effect of mGluR₅ blockade on seizure propensity in AD mouse models.

AGS was reduced or attenuated in fmr-1 KO, Tg2576 and FRAXAD mice after MPEP treatment. Mice that expressed elevated levels of mouse or human APP and Aβ had lower thresholds to AGS. mGluR5 blockade completely attenuated AGS in Tg2576 and reduced AGS in fmr-1^{-/-} and FRAXAD.

| Genotype | n | % WR | % AGS | % Death |
|---|---|---|---|---|
| Fmr-1 KO + | 14 | 14 | 14 | 0 |
| MPEP | | | | |
| FRAXAD + | 15 | 60 | 27 | 7 |
| MPEP | | | | |
| Tg2576 + MPEP | 16 | 0 | 0 | 0 |
| WT + MPEP | 19 | 0 | 0 | 0 |

Taken together, these results supported a role for FMRP in translational regulation (repression) of APP translation, which could be abrogated by mGluR₅ receptor activation, and conversely that mGluR₅ receptor inhibition promoted FMRP-mediated APP translation repression. Moreover, either overproduction or underproduction of APP (and presumably its proteolytic products) increased environmentally-induced seizures. These results supported the use of methods for blockading mGluR₅ by fenobam or MPEP to reduce APP/Aβ production and consequent phenotypes.

The invention is not intended to be limited to the disclosed embodiments of the invention. It should be understood that the foregoing disclosure emphasizes certain specific embodiments of the invention and that all modifications of alternatives equivalent thereto are within the spirit and scope of the invention as set forth in the appended claims.

We claim:

1. A method for reducing amyloid precursor protein (APP) production in a cell in a subject that has, or has a predisposition for developing, Alzheimer's disease, autism, epilepsy, Down's syndrome, or fragile X mental retardation syndrome (FXS), wherein the method comprises contacting the cell with a metabotropic glutamate receptor (mGluR₅) antagonist.

2. The method according to claim 1, wherein the amount of Aβ produced by the cell is reduced.

3. The method according to claim 1, wherein the mGluR₅ antagonist is fenobam or 2-methyl-6-(phenylethynyl)pyridine (MPEP).

4. A method of treating Alzheimer's disease, autism, epilepsy, Down's syndrome, or fragile X mental retardation syndrome (FXS) in a subject, comprising administering to the subject in need thereof a therapeutically-effective amount of an mGluR₅ antagonist.

5. The method according to claim 4, wherein the mGluR₅ antagonist is fenobam or MPEP.

6. The method according to claim 4, wherein the subject has Alzheimer's disease, autism or Down's syndrome.

7. A method of reducing seizure frequency in a subject that has Alzheimer's disease, autism, epilepsy, Down's syndrome, or fragile X mental retardation syndrome (FXS), the method comprising administering to the subject in need thereof a therapeutically-effective amount of an mGluR₅ antagonist.

8. The method according to claim 7, wherein the mGluR₅ antagonist is fenobam or MPEP.

9. The method according to claim 7, wherein the subject has Alzheimer's disease, autism, or Down's syndrome.

10. The method according to claim 7, wherein the subject has epilepsy.

11. The method according to any of claims 1, 4, or 7, wherein the subject is a mammal.

12. The method according to claim 11, wherein the mammal is a dog or cat.

13. The method according to claim 11, wherein the mammal is a human.

14. A method for detecting fragile X mental retardation syndrome (FXS) in a mammal, wherein the method comprises:
(a) obtaining a blood sample from the mammal; and
(b) detecting the levels of Aβ 1-42 in the blood sample of (a),
wherein a reduced level of Aβ₁₋₄₂ detected in (b) as compared to a control sample indicates that the mammal has FXS.

15. The method of claim 14, wherein the mammal is a human.

16. The method of claim 14, wherein the blood sample is blood plasma or blood serum.

17. The method of claim 14, wherein the reduced level of Aβ 1-42 detected in the blood sample of the mammal is at least two-fold as compared to the control sample.

18. A method for detecting fragile X mental retardation syndrome (FXS) in a mammal, wherein the method comprises:
(a) obtaining a blood sample from the mammal;
(b) subjecting the blood sample to a modified ELISA assay comprising steps of (i) incubating the blood sample for at least three hours with an insoluble substrate having at least one antibody specific for Aβ₁₋₄₂ bound thereto; (ii) removing the unbound blood sample from the substrate; and (iii) incubating a detection antibody with the substrate; and
(c) detecting the levels of Aβ₁₋₄₂ bound to the antibody specific for Aβ₁₋₄₂ on the substrate,
wherein a reduced level of Aβ₁₋₄₂ detected in (c) as compared to a control sample indicates that the mammal has FXS.


SEQUENCE LISTING (24 sequences)

SEQ ID NO: 1 - DNA, 17 bp, Artificial Sequence
  Synthetic PCR primer
ccgtggcacccttttgg

SEQ ID NO: 2 - DNA, 15 bp, Artificial Sequence
  Synthetic PCR primer
gggcgggcgtcaaca

SEQ ID NO: 3 - DNA, 26 bp, Artificial Sequence
  Synthetic PCR primer
cattaaatcagttatggttcctttgg

SEQ ID NO: 4 - DNA, 27 bp, Artificial Sequence
  Synthetic PCR primer
tcggcatgtattagctctagaattacc

SEQ ID NO: 5 - DNA, 18 bp, Artificial Sequence
  Synthetic PCR primer
ccttctcgtgcccgacaa

SEQ ID NO: 6 - DNA, 22 bp, Artificial Sequence
  Synthetic PCR primer
ccactgaagatgggtctcacaa

SEQ ID NO: 7 - DNA, 15 bp, Artificial Sequence
  Synthetic PCR primer
gccctgcggcatcga

SEQ ID NO: 8 - DNA, 18 bp, Artificial Sequence
  Synthetic PCR primer
tccacgctgtcgctttcc

SEQ ID NO: 9 - DNA, 21 bp, Artificial Sequence
  Synthetic PCR primer
ggtggagcggacacagactac

SEQ ID NO: 10 - DNA, 23 bp, Artificial Sequence
  Synthetic PCR primer
tacgcttcctcttcctcaacatc

SEQ ID NO: 11 - DNA, 24 bp, Artificial Sequence
  Synthetic PCR primer
gatgttgaggaagaggaagctgat

SEQ ID NO: 12 - DNA, 20 bp, Artificial Sequence
  Synthetic PCR primer
ctctcggtggcctcttcgta

SEQ ID NO: 13 - DNA, 24 bp, Artificial Sequence
  Synthetic PCR primer
gatgttgaggaagaggaagctgat

SEQ ID NO: 14 - DNA, 20 bp, Artificial Sequence
  Synthetic PCR primer
tggcagtgctggttgttctc

SEQ ID NO: 15 - DNA, 23 bp, Artificial Sequence
  Synthetic PCR primer
aagaggaggaagtggctgatgtt

SEQ ID NO: 16 - DNA, 20 bp, Artificial Sequence
  Synthetic PCR primer
ctctcggtggcctcttcgta

SEQ ID NO: 17 - DNA, 23 bp, Artificial Sequence
  Synthetic PCR primer
aagaggaggaagtggctgatgtt

SEQ ID NO: 18 - DNA, 20 bp, Artificial Sequence
  Synthetic PCR primer
tggcagtgctggttgttctc

SEQ ID NO: 19 - DNA, 24 bp, Artificial Sequence
  Synthetic PCR primer
gatgttgaggaagaggaagctgat

SEQ ID NO: 20 - DNA, 20 bp, Artificial Sequence
  Synthetic PCR primer
cggactcagtggtggttgtg

SEQ ID NO: 21 - DNA, 23 bp, Artificial Sequence
  Synthetic PCR primer
aagaggaggaagtggctgatgtt

SEQ ID NO: 22 - DNA, 20 bp, Artificial Sequence
  Synthetic PCR primer
cggactcagtggtggttgtg

SEQ ID NO: 23 - DNA, 30 bp, Artificial Sequence
  Synthetic PCR primer
gcctgaacttgaattaatatacaaatcagt

SEQ ID NO: 24 - DNA, 25 bp, Artificial Sequence
  Synthetic PCR primer
cagtacacaaaacccatgaatcatg